SEQUENCE LISTING (96 sequences)

SEQ ID NO: 1 - protein, 119 aa, Artificial Sequence
  Description of Artificial Sequence Synthetic polypeptide
QMQLVQSGSELKKPGASVKVSCKASGYTFTSYAMNWVRQAPGQGLEWMGWINTNTGNPTYAQGFTGRFVFSLDTSVSTAYLQISSLKAEDTAVYYCARFDRPRGAFDIWGQGTLVTVSS

SEQ ID NO: 2 - protein, 116 aa, Artificial Sequence
  Description of Artificial Sequence Synthetic polypeptide
QAVLTQPSSLSASPGASASLTCTLRSGINVGTYRIYWYQQKPGSPPQYLLRYKSDSDKQQGSGVPSRFSGSKDASANAGILLISGLQSEDEADYYCMIWHSSAWVFGGGTKLTVLG

SEQ ID NO: 3 - protein, 15 aa, Artificial Sequence
  Description of Artificial Sequence Synthetic peptide
GGGGSGGGGSGGGGS

SEQ ID NO: 4 - protein, 249 aa, Artificial Sequence
  Description of Artificial Sequence Synthetic polypeptide
QVQLVESGSELKKPGASVKVSCKASGYTFTNYAMNWVRQAPGQGLEWLGWINTNTGNPTYAQGFTGRFVFSLDTSVSTAYLQISSLKAEDTAVYYCARGSLGGTFDYWGQGTPVTVSSGGGGSGGGGSGGGGSQAVLTQPSSLSASPGASASLTCTLRSGINVGTYRIYWYQQKPGSPPQYLLRYKSDSDKHQDSGVPSRFSGSTDASANAGILLISGLQSEDEADYYCMIWHSRAWVFGGGTRGEIKR

SEQ ID NO: 5 - protein, 249 aa, Artificial Sequence
  Description of Artificial Sequence Synthetic polypeptide
QVQLVQSGAEVKKPGASVKVSCKASGYTFTSYGISWVRQAPGQGLEWMGWINTNTGNPTYAQGFTGRSVFSLDTSVSTAYLQISSLKAEDSAVYYCARHSDDGAFDIWGKGTMVTVSSGGGGSGGGGSGGGGSQAVLTQPSSLSASPGASASLTCTLRSGINVGTYRIYWYQQKPGSPPQYLLRYKSDSDKQQGSGVPSRFSGSKDASANAGTLLISGLQSEDEADYYCAIWHSSTWVFGGGTKVTVLG

SEQ ID NO: 6 - protein, 252 aa, Artificial Sequence
  Description of Artificial Sequence Synthetic polypeptide
QVQLVQSGAEVKKPGSSVKVSCKASGYTFADYAINWVRQAPGQGLEWMGWINTNTGNPTYAQGFTGRFVFSLDTSVSTAYLQISSLEAEDTAVYYCARHFSGYSLDAVDIWGQGTLVTVSSGGGGSGGGGSGGGGSQAVLTQPSSLSASPGTSASLTCTLRSDINVGAYRIYWYKHKAGSPPQFLLRFKSDSDNYRGSGVPGRFSGSKDASANAGILLISGLQSEDEADYYCAIWHSSTWVFGGGTKLTVLG

SEQ ID NO: 7 - protein, 250 aa, Artificial Sequence
  Description of Artificial Sequence Synthetic polypeptide
QMQLVQSGSELKKPGASVKVSCKASGYTFTSYAMNWVRQAPGQGLEWMGWINTNTGNPTYAQGFTGRFVFSLDTSVSTAYLQISSLKAEDTAVYYCARFDRPRGAFDIWGQGTLVTVSSGGGGSGGGGSGGGGSQAVLTQPSSLSASPGASASLTCTLRSGINVGTYRIYWYQQKPGSPPQYLLRYKSDSDKQQGSGVPSRFSGSKDASANAGILLISGLQSEDEADYYCMIWHSSAWVFGGGTKLTVLG

SEQ ID NO: 8 - protein, 252 aa, Artificial Sequence
  Description of Artificial Sequence Synthetic polypeptide
EVQLVESGSELKKPGASVKVSCKASGYTFTSYAINWVRQAPGQGLEWMGWINTNTGNPTYAQGFTGRFVFSLDTSVSTAYLQISSLKAEDTAVYYCARGYYDSSGYNFDYWGKGTLVTVSSGGGGSGGGGSGGGGSQAVLTQPSSLSASPGASASLTCTLRSGINVGTYRIYWYQQKPGSPPQYLLRYKSDSDKQQGSGVPSRFSGSKDASANAGILLISGLQSEDEADYYCMIWHSSAWVFGGGTKLTVLG

SEQ ID NO: 9 - protein, 256 aa, Artificial Sequence
  Description of Artificial Sequence Synthetic polypeptide
QMQLVQSGSELKRPGASVRVSCKTSGYNFNNYAISWVRQAPGQGLEWMGWINTNTGNPTYAQGFTGRFVFSLDTSVSTSYLQISSLEAEDTAVYYCARHRDSGSPSGDYYYMDVWGKGTLVTVSSGGGGSGGGGSGGGGSQAVLTQPSSLSASPGASASLTCTLRSGINVGTYRIYWYQQKPGSPPQYLLTYKSDSDKQQGSGVPSRFSGSKDASANAGILLISGLQSEDEADYYCMIWHSSAYVFGTGTKVTVLG

SEQ ID NO: 10 - protein, 248 aa, Artificial Sequence
  Description of Artificial Sequence Synthetic polypeptide
QVQLVQSGSELKKPGASVKVSCKASGYTFTSYAMNWVRQAPGQGLEWMGWINTNTGNPTYAQGFTGRFVFSLDTSVSTAYLQISSLKAEDTAVYYCARGAAGVFDIWGKGTTVTVSSGGGGSGGGGSGGGGSQAVLTQPSSLSASPGASASLTCTLRSGINVGTYNIYWYQQKPGSPPQFLLRYKSDSDKQQVSGVPSRFSGSKDGSANAGILLISGLQSEDEADYYCMIWHSSAWVFGGGTKLTVLG

SEQ ID NO: 11 - protein, 248 aa, Artificial Sequence
  Description of Artificial Sequence Synthetic polypeptide
EVQLVESGAEVKKPGASVKVSCKASGYTFTSYAMNWVRQAPGQGLEWMGWINTNTGNPTYAQGFTGRFVFSLDTSVSTAYLQISSLKAEDTAVYYCARQRDYRMDVWGQGTMVTVSSGGGGSGGGGSGGGGSQAVLTQPSSLSASPGASASLTCTLRSGINVGTYRIYWYQQKPGSPPQYLLRYKSDSDKQQASGVPSRFSGSKDASANAGILLISGLQSEDEADYYCLIWHNNAWVFGGGTKVTVLG

SEQ ID NO: 12 - protein, 246 aa, Artificial Sequence
  Description of Artificial Sequence Synthetic polypeptide
QVQLVQSGAEVKKPGASVKVSCKASGYSFTSYAMNWVRQAPGQGLEWMGWINTNTGNPMYAQGFTGRFVFSLDTSVSTAYLEVSSLKAEDTAVYFCARYNAMDVWGQGTTVTVSSGGGGSGGGGSGGGGSQAVLTQPSSLSASPGASASLTCTLRSGINVGTYRIYWYQQKPGSPPQYLLRYKSDSDKQQGSGVPSRFSGSKDASANAGILLISGLQSEDEADYYCMIWHSSAWVFGGGTKVTVLG

SEQ ID NO: 13 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - RNA, 16 nt, Artificial Sequence
  Description of Artificial Sequence Synthetic oligonucleotide
uuucuucaggacacag

SEQ ID NO: 18 - RNA, 16 nt, Artificial Sequence
  Description of Artificial Sequence Synthetic oligonucleotide
guccagaugucuuugc

SEQ ID NO: 19 - RNA, 40 nt, Artificial Sequence
  Description of Artificial Sequence Synthetic oligonucleotide
uuuuuccuguguccugaagaaagcaaagacaucuggacau

SEQ ID NO: 20 - protein, 6 aa, Artificial Sequence
  Description of Artificial Sequence Synthetic 6xHis tag
HHHHHH

SEQ ID NO: 21 - protein, 25 aa, Artificial Sequence
  Description of Artificial Sequence Synthetic peptide
QMQLVQSGSELKKPGASVKVSCKAS

SEQ ID NO: 22 - protein, 30 aa, Artificial Sequence
  Description of Artificial Sequence Synthetic polypeptide
QMQLVQSGSELKKPGASVKVSCKASGYTFT

SEQ ID NO: 23 - protein, 29 aa, Artificial Sequence
  Description of Artificial Sequence Synthetic peptide
QMQLVQSGSELKKPGASVKVSCKASGYTF

SEQ ID NO: 24 - protein, 7 aa, Artificial Sequence
  Description of Artificial Sequence Synthetic peptide
GYTFTSY

SEQ ID NO: 25 - protein, 10 aa, Artificial Sequence
  Description of Artificial Sequence Synthetic peptide
GYTFTSYAMN

SEQ ID NO: 26 - protein, 5 aa, Artificial Sequence
  Description of Artificial Sequence Synthetic peptide
SYAMN

SEQ ID NO: 27 - protein, 6 aa, Artificial Sequence
  Description of Artificial Sequence Synthetic peptide
TSYAMN

SEQ ID NO: 28 - protein, 8 aa, Artificial Sequence
  Description of Artificial Sequence Synthetic peptide
GYTFTSYA

SEQ ID NO: 29 - protein, 19 aa, Artificial Sequence
  Description of Artificial Sequence Synthetic peptide
AMNWVRQAPGQGLEWMGWI

SEQ ID NO: 30 - protein, 14 aa, Artificial Sequence
  Description of Artificial Sequence Synthetic peptide
WVRQAPGQGLEWMG

SEQ ID NO: 31 - protein, 11 aa, Artificial Sequence
  Description of Artificial Sequence Synthetic peptide
WVRQAPGQGLE

SEQ ID NO: 32 - protein, 17 aa, Artificial Sequence
  Description of Artificial Sequence Synthetic peptide
MNWVRQAPGQGLEWMGW

SEQ ID NO: 33 - protein, 6 aa, Artificial Sequence
  Description of Artificial Sequence Synthetic peptide
NTNTGN

SEQ ID NO: 34 - protein, 10 aa, Artificial Sequence
  Description of Artificial Sequence Synthetic peptide
WINTNTGNPT

SEQ ID NO: 35 - protein, 17 aa, Artificial Sequence
  Description of Artificial Sequence Synthetic peptide
WINTNTGNPTYAQGFTG

SEQ ID NO: 36 - protein, 13 aa, Artificial Sequence
  Description of Artificial Sequence Synthetic peptide
WMGWINTNTGNPT

SEQ ID NO: 37 - protein, 8 aa, Artificial Sequence
  Description of Artificial Sequence Synthetic peptide
INTNTGNP

SEQ ID NO: 38 - protein, 41 aa, Artificial Sequence
  Description of Artificial Sequence Synthetic polypeptide
PTYAQGFTGRFVFSLDTSVSTAYLQISSLKAEDTAVYYCAR

SEQ ID NO: 39 - protein, 39 aa, Artificial Sequence
  Description of Artificial Sequence Synthetic polypeptide
YAQGFTGRFVFSLDTSVSTAYLQISSLKAEDTAVYYCAR

SEQ ID NO: 40 - protein, 32 aa, Artificial Sequence
  Description of Artificial Sequence Synthetic polypeptide
RFVFSLDTSVSTAYLQISSLKAEDTAVYYCAR

SEQ ID NO: 41 - protein, 37 aa, Artificial Sequence
  Description of Artificial Sequence Synthetic polypeptide
YAQGFTGRFVFSLDTSVSTAYLQISSLKAEDTAVYYC

SEQ ID NO: 42 - protein, 38 aa, Artificial Sequence
  Description of Artificial Sequence Synthetic polypeptide
TYAQGFTGRFVFSLDTSVSTAYLQISSLKAEDTAVYYC

SEQ ID NO: 43 - protein, 10 aa, Artificial Sequence
  Description of Artificial Sequence Synthetic peptide
FDRPRGAFDI

SEQ ID NO: 44 - protein, 11 aa, Artificial Sequence
  Description of Artificial Sequence Synthetic peptide
ARFDRPRGAFD

SEQ ID NO: 45 - protein, 12 aa, Artificial Sequence
  Description of Artificial Sequence Synthetic peptide
ARFDRPRGAFDI

SEQ ID NO: 46 - protein, 11 aa, Artificial Sequence
  Description of Artificial Sequence Synthetic peptide
WGQGTLVTVSS

SEQ ID NO: 47 - protein, 12 aa, Artificial Sequence
  Description of Artificial Sequence Synthetic peptide
IWGQGTLVTVSS

SEQ ID NO: 48 - protein, 22 aa, Artificial Sequence
  Description of Artificial Sequence Synthetic peptide
QAVLTQPSSLSASPGASASLTC

SEQ ID NO: 49 - protein, 28 aa, Artificial Sequence
  Description of Artificial Sequence Synthetic peptide
QAVLTQPSSLSASPGASASLTCTLRSGI

SEQ ID NO: 50 - protein, 25 aa, Artificial Sequence
  Description of Artificial Sequence Synthetic peptide
QAVLTQPSSLSASPGASASLTCTLR

SEQ ID NO: 51 - protein, 14 aa, Artificial Sequence
  Description of Artificial Sequence Synthetic peptide
TLRSGINVGTYRIY

SEQ ID NO: 52 - protein, 10 aa, Artificial Sequence
  Description of Artificial Sequence Synthetic peptide
NVGTYRIYWY

SEQ ID NO: 53 - protein, 9 aa, Artificial Sequence
  Description of Artificial Sequence Synthetic peptide
SGINVGTYR

SEQ ID NO: 54 - protein, 15 aa, Artificial Sequence
  Description of Artificial Sequence Synthetic peptide
WYQQKPGSPPQYLLR

SEQ ID NO: 55 - protein, 9 aa, Artificial Sequence
  Description of Artificial Sequence Synthetic peptide
QQKPGSPPQ

SEQ ID NO: 56 - protein, 17 aa, Artificial Sequence
  Description of Artificial Sequence Synthetic peptide
IYWYQQKPGSPPQYLLR

SEQ ID NO: 57 - protein, 11 aa, Artificial Sequence
  Description of Artificial Sequence Synthetic peptide
YKSDSDKQQGS

SEQ ID NO: 58 - protein, 14 aa, Artificial Sequence
  Description of Artificial Sequence Synthetic peptide
YLLRYKSDSDKQQG

SEQ ID NO: 59 - protein, 6 aa, Artificial Sequence
  Description of Artificial Sequence Synthetic peptide
YKSDSD

SEQ ID NO: 60 - protein, 34 aa, Artificial Sequence
  Description of Artificial Sequence Synthetic polypeptide
GVPSRFSGSKDASANAGILLISGLQSEDEADYYC

SEQ ID NO: 61 - protein, 35 aa, Artificial Sequence
  Description of Artificial Sequence Synthetic polypeptide
SGVPSRFSGSKDASANAGILLISGLQSEDEADYYC

SEQ ID NO: 62 - protein, 39 aa, Artificial Sequence
  Description of Artificial Sequence Synthetic polypeptide
KQQGSGVPSRFSGSKDASANAGILLISGLQSEDEADYYC

SEQ ID NO: 63 - protein, 9 aa, Artificial Sequence
  Description of Artificial Sequence Synthetic peptide
MIWHSSAWV

SEQ ID NO: 64 - protein, 8 aa, Artificial Sequence
  Description of Artificial Sequence Synthetic peptide
MIWHSSAW

SEQ ID NO: 65 - protein, 11 aa, Artificial Sequence
  Description of Artificial Sequence Synthetic peptide
FGGGTKLTVLG

SEQ ID NO: 66 - protein, 12 aa, Artificial Sequence
  Description of Artificial Sequence Synthetic peptide
VFGGGTKLTVLG

SEQ ID NO: 67 - protein, 5 aa, Artificial Sequence
  Description of Artificial Sequence Synthetic peptide
NYAMN

SEQ ID NO: 68 - protein, 5 aa, Artificial Sequence
  Description of Artificial Sequence Synthetic peptide
SYGIS

SEQ ID NO: 69 - protein, 5 aa, Artificial Sequence
  Description of Artificial Sequence Synthetic peptide
DYAIN

SEQ ID NO: 70 - protein, 5 aa, Artificial Sequence
  Description of Artificial Sequence Synthetic peptide
NYAIS

SEQ ID NO: 71 - protein, 5 aa, Artificial Sequence
  Description of Artificial Sequence Synthetic peptide
SYAIN

SEQ ID NO: 72 - protein, 17 aa, Artificial Sequence
  Description of Artificial Sequence Synthetic peptide
WINTNTGNPMYAQGFTG

SEQ ID NO: 73 - protein, 9 aa, Artificial Sequence
  Description of Artificial Sequence Synthetic peptide
GSLGGTFDY

SEQ ID NO: 74 - protein, 9 aa, Artificial Sequence
  Description of Artificial Sequence Synthetic peptide
HSDDGAFDI

SEQ ID NO: 75 - protein, 12 aa, Artificial Sequence
  Description of Artificial Sequence Synthetic peptide
HFSGYSLDAVDI

SEQ ID NO: 76 - protein, 16 aa, Artificial Sequence
  Description of Artificial Sequence Synthetic peptide
HRDSGSPSGDYYYMDV

SEQ ID NO: 77 - protein, 12 aa, Artificial Sequence
  Description of Artificial Sequence Synthetic peptide
GYYDSSGYNFDY

SEQ ID NO: 78 - protein, 8 aa, Artificial Sequence
  Description of Artificial Sequence Synthetic peptide
GAAGVFDI

SEQ ID NO: 79 - protein, 8 aa, Artificial Sequence
  Description of Artificial Sequence Synthetic peptide
QRDYRMDV

SEQ ID NO: 80 - protein, 6 aa, Artificial Sequence
  Description of Artificial Sequence Synthetic peptide
YNAMDV

SEQ ID NO: 81 - protein, 14 aa, Artificial Sequence
  Description of Artificial Sequence Synthetic peptide
TLRSDINVGAYRIY

SEQ ID NO: 82 - protein, 14 aa, Artificial Sequence
  Description of Artificial Sequence Synthetic peptide
TLRSGINVGTYNIY

SEQ ID NO: 83 - protein, 11 aa, Artificial Sequence
  Description of Artificial Sequence Synthetic peptide
YKSDSDKHQDS

SEQ ID NO: 84 - protein, 11 aa, Artificial Sequence
  Description of Artificial Sequence Synthetic peptide
FKSDSDNYRGS

SEQ ID NO: 85 - protein, 11 aa, Artificial Sequence
  Description of Artificial Sequence Synthetic peptide
YKSDSDKQQVS

SEQ ID NO: 86 - protein, 11 aa, Artificial Sequence
  Description of Artificial Sequence Synthetic peptide
YKSDSDKQQAS

SEQ ID NO: 87 - protein, 9 aa, Artificial Sequence
  Description of Artificial Sequence Synthetic peptide
MIWHSRAWV

SEQ ID NO: 88 - protein, 9 aa, Artificial Sequence
  Description of Artificial Sequence Synthetic peptide
AIWHSSTWV

SEQ ID NO: 89 - protein, 9 aa, Artificial Sequence
  Description of Artificial Sequence Synthetic peptide
MIWHSSAYV

SEQ ID NO: 90 - protein, 9 aa, Artificial Sequence
  Description of Artificial Sequence Synthetic peptide
LIWHNNAWV

SEQ ID NO: 91 - protein, 9 aa, Artificial Sequence
  Description of Artificial Sequence Synthetic peptide
SGGGERTHK

SEQ ID NO: 92 - protein, 9 aa, Artificial Sequence
  Description of Artificial Sequence Synthetic peptide
SGGGATEHK

SEQ ID NO: 93 - protein, 9 aa, Artificial Sequence
  Description of Artificial Sequence Synthetic peptide
SGGGDIHRK

SEQ ID NO: 94 - protein, 6 aa, Artificial Sequence
  Description of Artificial Sequence Synthetic peptide
ARPDGG

SEQ ID NO: 95 - protein, 9 aa, Artificial Sequence
  Description of Artificial Sequence Synthetic peptide
SGGGGGGGK

SEQ ID NO: 96 - protein, 6 aa, Artificial Sequence
  Description of Artificial Sequence Synthetic peptide
GGPDGG

(19) United States
(12) Patent Application Publication
(10) Pub. No.: US 2024/0002536 A1
(43) Pub. Date: Jan. 4, 2024

(54) Antibodies and Single-Chain Variable Region Proteins Recognizing Click Products

(71) Applicant: The General Hospital Corporation, Boston, MA (US)

(72) Inventors: James T. Kurnick, Quincy, MA (US); Ian Seymour Dunn, Beverly, MA (US); Matthew M. Lawler, Madison, WI (US)

(21) Appl. No.: 18/035,982

(22) PCT Filed: Nov. 5, 2021
(86) PCT No.: PCT/US2021/058322

§ 371 Date: May 9, 2023

Related U.S. Application Data
(60) Provisional application No. 63/111,450, filed on Nov. 9, 2020.

(51) Int. Cl. C07K 16/44; G01N 33/53

(52) U.S. Cl. CPC C07K 16/44; G01N 33/5308; C07K 2317/92; C07K 2317/21; C07K 2317/33; C07K 2317/622

(57) ABSTRACT

The present disclosure is directed, at least in part, to click-product binding molecules for recognition of templated assembly products defined by bioorthogonal click groups. Such molecules can be used for therapeutic and diagnostic purposes, including kits for the same.

### CROSS-REFERENCE TO RELATED APPLICATIONS

This application claims priority to U.S. Provisional Patent Application No. 63/111,450, filed Nov. 9, 2020. The contents of this application are incorporated herein by reference in its entirety.

### FIELD OF THE INVENTION

The present disclosure is directed, at least in part, to click-product binding molecules for recognition of templated assembly products defined by bioorthogonal click groups. Such molecules can be used for therapeutic and diagnostic purposes, including kits for the same.

### BACKGROUND

The process of Templated Assembly by Proximity-Enhanced Reactivity (TAPER) provides for the acquisition of function arising from the reaction between two otherwise inert components, through a template-dependent assembly effect (see, e.g., WO2014197547). Although the function of the assembled moiety can be direct, such as producing cytotoxic effects in target cells, a frequent aim is for the assembly of a structure that is recognizable by components of the immune system, which in turn mediate the desired final functional outcomes.

### SUMMARY

Provided herein are click product binding molecules that specifically bind to a click product of structure (1):

[A]-<azide-dibenzocyclooctyne(DBCO)>-[B] (1),
where [A] and [B] comprise distinct sequences, e.g., distinct tetrapeptide, pentapeptide, hexapeptide, or heptapeptide sequences, wherein the antibody comprises a heavy chain variable region (VH) from an antibody described herein, e.g., antibody 18, 36, 44, 51, 64, 65, 69, 72, or 83 as described herein, e.g., Ab 51, e.g., comprising a VH complementarity determining region (CDR)1 comprising an amino acid sequence set forth in Table 1A, a VH CDR2 comprising an amino acid sequence set forth in Table 1A, and a VH CDR3 comprising an amino acid sequence set forth in Table 1A; and a light chain variable region (VL) comprising a VL CDR1 from Ab 51 as described herein, e.g., comprising an amino acid sequence set forth in Table 1B a VL CDR2 comprising an amino acid sequence set forth in Table 1B, and a VL CDR3 comprising an amino acid sequence set forth in Table 1B (preferably wherein the CDRs are all from the same CDR definition).

In some embodiments, the VH comprises an amino acid sequence having at least 80% sequence identity to the amino acid sequence set forth in SEQ ID NO: 1, and/or the VL comprises an amino acid sequence having at least 80% sequence identity to the amino acid sequence set forth in SEQ ID NO:2. In some embodiments, the VH comprises an amino acid sequence having at least 80% sequence identity to the amino acid sequence set forth in SEQ ID NO:1, and the VL comprises an amino acid sequence having at least 80% sequence identity to the amino acid sequence set forth in SEQ ID NO:2.

In some embodiments, the VH comprises the amino acid sequence set forth in SEQ ID NO: 1, and/or the VL comprises the amino acid sequence set forth in SEQ ID NO:2. In some embodiments, the VH comprises the amino acid sequence set forth in SEQ ID NO: 1, and the VL comprises the amino acid sequence set forth in SEQ ID NO:2.

In some embodiments, the VH consists of the amino acid sequence set forth in SEQ ID NO: 1, and/or the VL consists of the amino acid sequence set forth in SEQ ID NO:2.

In some embodiments, the click product binding molecule comprises a heavy chain and a light chain and optionally an Fc region, preferably a human IgG1 Fc.

In some embodiments, the click product binding molecule is a single chain antibody molecule (scFv).

Also provided herein are single chain antibody molecule (scFv) that specifically binds to a click product of structure (1):

[A]-<azide-DBCO>-[B] (1),
where [A] and [B] comprise distinct sequences, e.g., distinct tetrapeptide, pentapeptide, hexapeptide, or heptapeptide sequences, the scFv comprising:
a VH consisting of the amino acid sequence set forth in SEQ ID NO:1 and a VL consisting of the amino acid sequence set forth in SEQ ID NO:2, or an amino acid sequence set forth in any one of SEQ ID NOs:4-12.

Further, provided herein are polynucleotides comprising a nucleic acid sequence encoding a click product binding molecule, e.g., an scFv, as described herein.

Also provided are polynucleotides comprising a nucleic acid sequence encoding a VH comprising the amino acid sequence set forth in SEQ ID NO: 1, and polynucleotides comprising a nucleic acid sequence encoding VL comprising the amino acid sequence set forth in SEQ ID NO: 2.

In some embodiments, the nucleic acid sequence is operably linked to a promoter.

Additionally provided are polynucleotides comprising a first nucleic acid sequence encoding a VH comprising the amino acid sequence set forth in SEQ ID NO: 1 and a second nucleic acid sequence encoding a VL comprising the amino acid sequence set forth in SEQ ID NO:2.

In some embodiments, the first nucleic acid sequence is operably linked to a first promoter, and the second nucleic acid sequence is operably linked to a second promoter.

Further provided herein are vectors comprising a polynucleotide as described herein, as well as host cells comprising the polynucleotides or vectors.

Also provided herein are pharmaceutical compositions comprising the click product binding molecules, e.g., the scFv, as described herein, and a pharmaceutically acceptable carrier or diluent.

Additionally provided herein are methods of making the click product binding molecules, e.g., the ScFv, as described herein; the methods comprise culturing the host cells described herein and isolating the click product binding molecule or scFv.

In some embodiments, the methods include formulating the antibody into a sterile pharmaceutical composition.

Further, provided herein are methods of detecting formation of a click product of structure (1):

[A]-<azide-DBCO>-[B] (1),
where [A] and [B] comprise distinct sequences, e.g., distinct tetrapeptide, pentapeptide, hexapeptide, or heptapeptide sequences, in a sample, the method comprising contacting the sample with the click product binding molecules, e.g., the scFv, as described herein, and detecting binding of the click product binding molecule or scFv to the sample.

In some embodiments, the sample comprises: haplomer A comprising at least a first sequence and an azide moiety; haplomer B comprising at least a second sequence and DBCO moiety; wherein the click product is produced is produced when haplomer A is in proximity with haplomer B. Preferably the first and second sequences comprise at least distinct tetrapeptide, pentapeptide, hexapeptide, or heptapeptide sequences.

In some embodiments, haplomer A and haplomer B bind to the same target. In some embodiments, haplomer A and haplomer B bind to two different targets, wherein the two different targets can interact in the sample.

The methods can also include isolating and optionally purifying and/or identifying the click products.

The samples can include tissues, tissue homogenate, cells, cell lysates, and other biologically-derived samples, including any biological fluids such as blood, serum, plasma, urine, semen, tears, ascites, sputum, cerebrospinal fluid, and so on.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF THE DRAWINGS

FIG. 1. General requirements for click-product recognition for implementation of TAPER, where successful binders must recognize the click product, but NOT the precursors.

FIG. 2. Modularity of TAPER process as applicable to anti-click product molecules.

FIG. 3. Chosen azide/DBCO Peptide Set for scFv library screen.

FIG. 4. Mass spectrometric analysis of Az3db click reaction for scFv library selection.

FIG. 5. Structures of the click reaction products between azide and DBCO groups.

FIG. 6. Specificity of target click peptide recognition by two selected scFvs (1 S, 2S).

FIG. 7. Examples of competition tests for scFv relative binding affinities.

FIG. 8. Comparing scFv ELISA response curve slopes: ratios of slopes without competitor (Az3db1 plate target): slopes with soluble competitors.

FIG. 9. scFv response curve slopes for test modified click peptides relative the original selection peptide Az3db1.

FIG. 10. Relative responses of scFv 4S and full antibody version of 4S (IgG1-51) towards selection target Az3db1 in ELISA assay.

FIG. 11. Novel gelshift assay for demonstration of binding of Az3db1 by IgG1-51 antibody. A, Schematic depiction of gelshift process exploiting the tetravalency of streptavidin (SA); B, Tris-glycine gel demonstration of antibody-mediated gelshift.

FIG. 12. Affinity K_{D} determination for IgG1-51 binding of Az3db1 by ELISA method.

FIG. 13. Binding of scFv 4S to azide-DBCO ‘half peptide’ db1.

FIG. 14. Treating HeLa and Jurkat cells (metabolically labeled with AzNAM, bearing surface azides) with DBCO-bearing db1 ‘half-peptide’, and probing with 4S scFv (anti-6H-Alexa-Fluor secondary).

FIG. 15. Treating AzNAM-labeled Jurkat cells with DBCO-bearing db1 ‘half-peptide’, and probing with IgG1-51 antibody (anti-human kappa-FITC secondary).

FIG. 16A. Components of system for biomarker-selective detection or therapy based on haplomer-mediated click product binding molecule binding. The biomarker molecule is shown as having two binding sites, A and B.

FIG. 16B. Haplomers binding to biomarker and click product formation. Complementary pairs of Haplomers bind at adjacent sites A and B on a single biomarker, driving formation of a click product. The click product forms selectively in the presence of the biomarker.

FIG. 16C. Click Product binding molecules binding to click products formed by the paired haplomers binding to the biomarker.

FIG. 17A. Components of system for biomarker interaction detection or therapy based on haplomer-mediated click product binding molecule binding.

FIG. 17B. Haplomers binding to different biomarkers and click product formation driven by biomarker interaction. Complementary pairs of Haplomers bind to sites on different biomarkers. If Biomarker A and Biomarker B interact, bound complementary Haplomers are brought into proximity, driving formation of click product. The click product thus forms selectively in the presence of Biomarker A-Biomarker B interactions.

FIG. 17C. Click product binding molecule binding at interaction site. The click product binding molecule binds to the click product formed by the Haplomers at the Biomarker A-Biomarker B interaction site.

FIG. 18A. Components of system for screening libraries of haplomers for biomarker binding based on click product binding molecule detection of library hits.

FIG. 18B. Haplomers library constituents binding to biomarker and click product formation. Libraries of complementary haplomers are mixed in the presence of a biomarker. If complementary library constituents bind at adjacent sites on the biomarker, click groups on bound complementary Haplomers are brought into proximity, driving formation of click product.

FIG. 18C. Click Product binding molecule facilitating identification of biomarker-binding hits from haplomers libraries. The click product binding molecule binds to click products formed by Haplomers bound to the biomarker. Antibody-mediated isolation and analysis of biomarker-bound Haplomers facilitates identification of hits from libraries.

### DETAILED DESCRIPTION

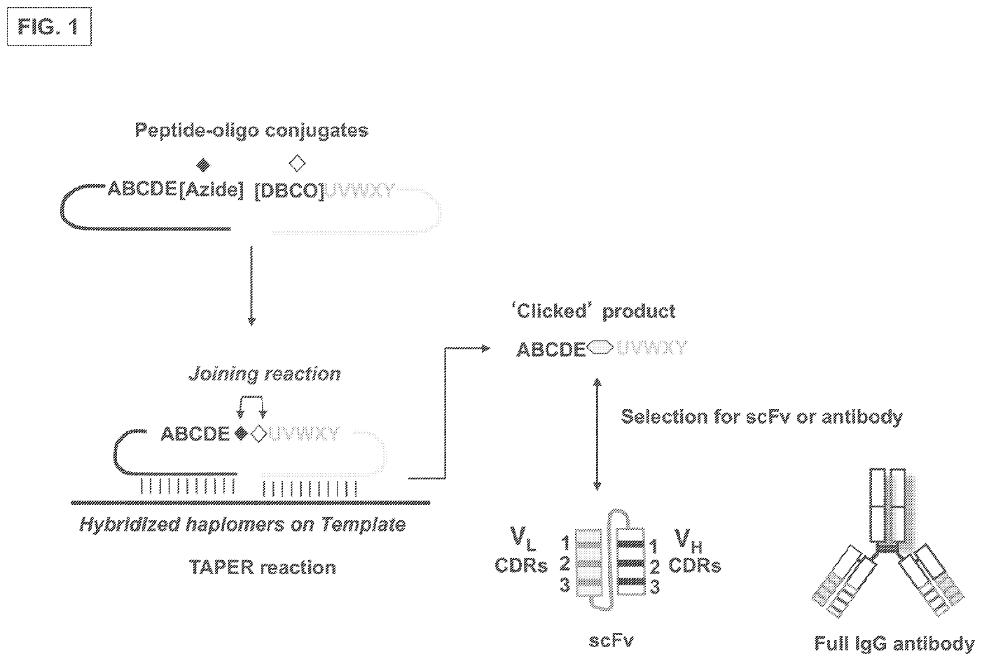

The ability to generate ‘split epitopes’ is of considerable practical and therapeutic significance. This refers to the generation of separate structures that can be assembled on a template to enable antibody recognition, where in the absence of assembly no such recognition can occur (see, e.g., WO2019032942). An optimal split-epitope system would be assembled through template-dependent bio-orthogonal and highly efficient processes, which can approached with specific examples of ‘click’ chemistry. From first principles, an antibody against a specific peptide epitope might be adapted to binding a peptide derived from the product of click reactions between two shorter peptides comprising a portion of the epitope, but in practice the bulkiness of available click groups places strong constraints on the ability to do this. Alternatively, in a reverse approach, an antibody (or an alternative binding agent, including antibody-derived simpler structures such as scFv proteins) could be selected for binding affinity towards a defined peptide click product. Where such a recognition agent directly binds a clicked target, its ‘split’ components are immediately defined as the click group-bearing peptide fragments from which the target was derived in the first place. Where such fragments can be equipped with nucleic acid sequence tags for allowing hybridization to a common template, a click chemistry-based split-epitope system can be effected, as depicted in FIG. 1. In this case, the click moieties are azide and dibenzocyclooctyne (DBCO), which produce a click peptide of structure (1):

[A]-<azide-DBCO>-[B] (1),
where [A] and [B] represent distinct tetrapeptide sequences.

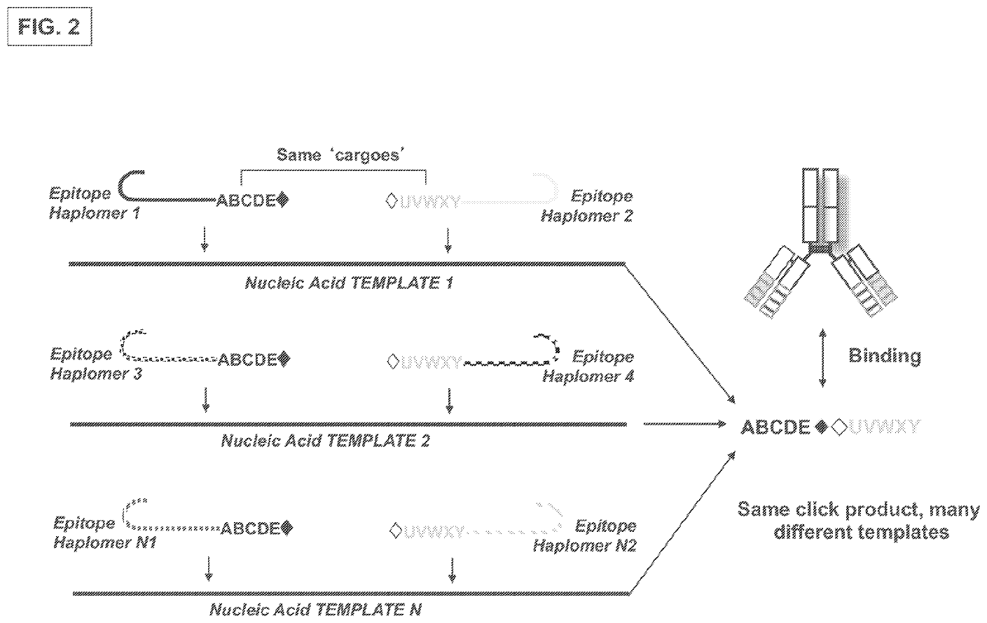

Using the general modularity of TAPER (see, e.g., WO2019032942), click peptide fragments can be linked to an indefinite number of separate sequences to allow hybridization to (in principle) any target template sequence (FIG. 2).

Thus described herein is a click-based split epitope system, as well as click-product binding molecules that bind specifically to assembled click products.

The binding molecules (also referred to herein as binding agents) described herein bind specifically to assembled click products, regardless of the sequence on either side. In some embodiments, a binding agent comprises an antibody or an antigen-binding fragment thereof. In some embodiments, a binding agent is an antibody or an antigen-binding fragment thereof. In some embodiments, a binding agent comprises an alternative protein scaffold or artificial scaffold (e.g., a non-immunoglobulin backbone). In some embodiments, a binding agent is a fusion protein comprising an antigen-binding site. In some embodiments, a binding agent is a bispecific or multispecific molecule comprising at least one antigen-binding site.

The term “antibody” as used herein refers to an immunoglobulin molecule that recognizes and binds a target through at least one antigen-binding site. “Antibody” is used herein in the broadest sense and encompasses various antibody structures, including but not limited to, polyclonal antibodies, recombinant antibodies, monoclonal antibodies, chimeric antibodies, humanized antibodies, human antibodies, bispecific antibodies, multispecific antibodies, diabodies, tribodies, tetrabodies, single chain Fv (scFv) antibodies, and antibody fragments as long as they exhibit the desired antigen-binding activity.

The term “intact antibody” or “full-length antibody” refers to an antibody having a structure substantially similar to a native antibody structure. This includes, for example, an antibody comprising two light chains each comprising a variable region and a light chain constant region (CL) and two heavy chains each comprising a variable region and at least heavy chain constant regions CH1, CH2, and CH3. Generally, an intact antibody includes a hinge region (or a portion thereof) between the CH1 and CH2 regions.

The term “antibody fragment” as used herein refers to a molecule other than an intact antibody that comprises a portion of an antibody and generally an antigen-binding site. Examples of antibody fragments include, but are not limited to, Fab, Fab′, F(ab′)2, Fv, single chain antibody molecules (e.g., scFv), sc(Fv)2, disulfide-linked scFv (dsscFv), diabodies, tribodies, tetrabodies, minibodies, dual variable domain antibodies (DVD), single variable domain antibodies (e.g., camelid antibodies), and multispecific antibodies formed from antibody fragments.

The term “monoclonal antibody” as used herein refers to a substantially homogenous antibody population involved in the highly specific recognition and binding of a single antigenic determinant or epitope. The term “monoclonal antibody” encompasses intact and full-length monoclonal antibodies as well as antibody fragments (e.g., Fab, Fab′, F(ab′)₂, Fv), single chain antibodies (e.g., scFv), fusion proteins comprising an antibody fragment, and any other modified immunoglobulin molecule comprising at least one antigen-binding site. Furthermore, “monoclonal antibody” refers to such antibodies made by any number of techniques, including but not limited to, hybridoma production, phage library display, recombinant expression, and transgenic animals.

The term “chimeric antibody” refers to an antibody in which a portion of the heavy and/or light chain is derived from a first source or species, while the remainder of the heavy and/or light chain is derived from a different source or species.

The term “humanized antibody” as used herein refers to an antibody that comprises a human heavy chain variable region and a light chain variable region wherein the native CDR amino acid residues are replaced by residues from corresponding CDRs from a nonhuman antibody (e.g., mouse, rat, rabbit, or nonhuman primate), wherein the nonhuman antibody has the desired specificity, affinity, and/or activity. In some embodiments, one or more framework region amino acid residues of the human heavy chain or light chain variable regions are replaced by corresponding residues from nonhuman antibody. Furthermore, humanized antibodies can comprise amino acid residues that are not found in the human antibody or in the nonhuman antibody. In some embodiments, these modifications are made to further refine and/or optimize antibody characteristics. In some embodiments, the humanized antibody comprises at least a portion of an immunoglobulin constant region (e.g., CH1, CH2, CH3, Fc, and/or hinge region), typically that of a human immunoglobulin.

The term “human antibody” as used herein refers to an antibody that possesses an amino acid sequence that corresponds to an antibody produced by a human and/or an antibody that has been made using any of the techniques that are known to those of skill in the art for making human antibodies. These techniques include, but not limited to, phage display libraries, yeast display libraries, transgenic animals, recombinant protein production, and B-cell hybridoma technology.

The terms “epitope” and “antigenic determinant” are used interchangeably herein and refer to that portion of an antigen or target capable of being recognized and bound by a particular antibody. When the antigen or target is a polypeptide, epitopes can be formed both from contiguous amino acids and noncontiguous amino acids juxtaposed by tertiary folding of the protein. Epitopes formed from contiguous amino acids (also referred to as linear epitopes) are typically retained upon protein denaturing, whereas epitopes formed by tertiary folding (also referred to as conformational epitopes) are typically lost upon protein denaturing. An epitope typically includes at least 3, and more usually, at least 5, 6, 7, or 8-10 amino acids in a unique spatial conformation. Epitopes can be predicted using any one of a large number of software bioinformatic tools available on the internet. X-ray crystallography may be used to characterize an epitope on a target protein by analyzing the amino acid residue interactions of an antigen/antibody complex.

The term “specifically binds” as used herein refers to an agent (e.g., an antibody) that interacts more frequently, more rapidly, with greater duration, with greater affinity, or with some combination of the above to a particular antigen, epitope, protein, or target molecule than with alternative substances. A binding agent (e.g. antibody) that specifically binds an antigen can be identified, for example, by immunoassays, ELISAs, surface plasmon resonance (SPR), or other techniques known to those of skill in the art. A binding agent that specifically binds an antigen can bind the target antigen at a higher affinity than its affinity for a different antigen. The different antigen can be a related antigen. In some embodiments, a binding agent that specifically binds an antigen can bind the target antigen with an affinity that is at least 20 times greater, at least 30 times greater, at least 40 times greater, at least 50 times greater, at least 60 times greater, at least 70 times greater, at least 80 times greater, at least 90 times greater, or at least 100 times greater, than its affinity for a different antigen. In some embodiments, a binding agent that specifically binds a particular antigen binds a different antigen at such a low affinity that binding cannot be detected using an assay described herein or otherwise known in the art. In some embodiments, affinity is measured using SPR technology in a Biacore system as described herein or as known to those of skill in the art.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification. Also included within the definition are, for example, polypeptides containing one or more analogs of an amino acid, including but not limited to, unnatural amino acids, as well as other modifications known in the art. It is understood that, because the polypeptides of this disclosure may be based upon antibodies, the term “polypeptide” encompasses polypeptides as a single chain and polypeptides of two or more associated chains.

The terms “polynucleotide” and “nucleic acid” and “nucleic acid molecule” are used interchangeably herein and refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase.

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity may be measured using sequence comparison software or algorithms or by visual inspection. Various algorithms and software that may be used to obtain alignments of amino acid or nucleotide sequences are well-known in the art. These include, but are not limited to, BLAST, ALIGN, Megalign, BestFit, GCG Wisconsin Package, and variants thereof. In some embodiments, two nucleic acids or polypeptides of the disclosure are substantially identical, meaning they have at least 70%, at least 75%, at least 80%, at least 85%, at least 90%, and in some embodiments at least 95%, 96%, 97%, 98%, 99% nucleotide or amino acid residue identity, when compared and aligned for maximum correspondence, as measured using a sequence comparison algorithm or by visual inspection. In some embodiments, identity exists over a region of the sequences that is at least about 10, at least about 20, at least about 20-40, at least about 40-60 nucleotides or amino acid residues, at least about 60-80 nucleotides or amino acid residues in length or any integral value there between. In some embodiments, identity exists over a longer region than 60-80 nucleotides or amino acid residues, such as at least about 80-100 nucleotides or amino acid residues, and in some embodiments the sequences are substantially identical over the full length of the sequences being compared, for example, (i) the coding region of a nucleotide sequence or (ii) an amino acid sequence.

The phrase “conservative amino acid substitution” as used herein refers to a substitution in which one amino acid residue is replaced with another amino acid residue having a similar side chain. Families of amino acid residues having similar side chains have been generally defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is considered to be a conservative substitution. Generally, conservative substitutions in the sequences of polypeptides and/or antibodies do not abrogate the binding of the polypeptide or antibody to the target binding site. Methods of identifying nucleotide and amino acid conservative substitutions that do not eliminate binding are well-known in the art.

The term “vector” as used herein means a construct that is capable of delivering, and usually expressing, one or more gene(s) or sequence(s) of interest in a host cell. Examples of vectors include, but are not limited to, viral vectors, naked DNA or RNA expression vectors, plasmid, cosmid, or phage vectors, DNA or RNA expression vectors associated with cationic condensing agents, and DNA or RNA expression vectors encapsulated in liposomes.

The term “isolated” as used herein refers to a polypeptide, soluble protein, antibody, polynucleotide, vector, cell, or composition that is in a form not found in nature. An “isolated” antibody is substantially free of material from the cellular source from which it is derived. In some embodiments, isolated polypeptides, soluble proteins, antibodies, polynucleotides, vectors, cells, or compositions are those that have been purified to a degree that they are no longer in a form in which they are found in nature. In some embodiments, a polypeptide, soluble protein, antibody, polynucleotide, vector, cell, or composition that is isolated is substantially pure. A polypeptide, soluble protein, antibody, polynucleotide, vector, cell, or composition can be isolated from a natural source (e.g., tissue) or from a source such as an engineered cell line.

The term “substantially pure” as used herein refers to material that is at least 50% pure (i.e., free from contaminants), at least 90% pure, at least 95% pure, at least 98% pure, or at least 99% pure.

The term “subject” refers to any animal (e.g., a mammal), including, but not limited to, humans, non-human primates, canines, felines, rabbits, rodents, and the like.

The term “pharmaceutically acceptable” as used herein refers to a substance approved or approvable by a regulatory agency or listed in the U.S. Pharmacopeia, European Pharmacopeia, or other generally recognized pharmacopeia for use in animals, including humans.

The terms “pharmaceutically acceptable excipient, carrier, or adjuvant” or “acceptable pharmaceutical carrier” as used herein refer to an excipient, carrier, or adjuvant that can be administered to a subject, together with at least one therapeutic agent (e.g., an antibody), and that is generally safe, non-toxic, and has no effect on the pharmacological activity of the therapeutic agent. In general, those of skill in the art and the U.S. FDA consider a pharmaceutically acceptable excipient, carrier, or adjuvant to be an inactive ingredient of any formulation.

The term “pharmaceutical formulation” or “pharmaceutical composition” as used herein refers to a preparation that is in such form as to permit the biological activity of the agent (e.g., an antibody) to be effective. A pharmaceutical formulation or composition generally comprises additional components, such as a pharmaceutically acceptable excipient, carrier, adjuvant, buffers, etc.

The term “effective amount” or “therapeutically effective amount” as used herein refers to the amount of an agent (e.g., an antibody) that is sufficient to reduce and/or ameliorate the severity and/or duration of (i) a disease, disorder or condition in a subject, and/or (ii) a symptom in a subject. The term also encompasses an amount of an agent necessary for the (i) reduction or amelioration of the advancement or progression of a given disease, disorder, or condition, (ii) reduction or amelioration of the recurrence, development, or onset of a given disease, disorder, or condition, and/or (iii) the improvement or enhancement of the prophylactic or therapeutic effect(s) of another agent or therapy (e.g., an agent other than the binding agents provided herein).

The term “therapeutic effect” as used herein refers to the effect and/or ability of an agent (e.g., an antibody) to reduce and/or ameliorate the severity and/or duration of (i) a disease, disorder, or condition in a subject, and/or (ii) a symptom in a subject. The term also encompasses the ability of an agent to (i) reduce or ameliorate the advancement or progression of a given disease, disorder, or condition, (ii) reduce or ameliorate the recurrence, development, or onset of a given disease, disorder, or condition, and/or (iii) to improve or enhance the prophylactic or therapeutic effect(s) of another agent or therapy (e.g., an agent other than the binding agents provided herein).

The term “treat” or “treatment” or “treating” or “to treat” or “alleviate” or alleviation” or “alleviating” or “to alleviate” as used herein refers to both (1) therapeutic measures that aim to cure, slow down, lessen symptoms of, and/or halt progression of a pathologic condition or disorder and (2) prophylactic or preventative measures that aim to prevent or slow the development of a targeted pathologic condition or disorder. Thus, those in need of treatment include those already with the disorder, those at risk of having/developing the disorder, and those in whom the disorder is to be prevented.

The term “prevent” or “prevention” or “preventing” as used herein refers to the partial or total inhibition of the development, recurrence, onset, or spread of a disease, disorder, or condition, or a symptom thereof in a subject.

The term “immune response” as used herein includes responses from both the innate immune system and the adaptive immune system. It includes both cell-mediated and/or humoral immune responses. It includes both T-cell and B-cell responses, as well as responses from other cells of the immune system such as natural killer (NK) cells, monocytes, macrophages, etc.

As used herein, reference to “about” or “approximately” a value or parameter includes (and describes) embodiments that are directed to that value or parameter. For example, a description referring to “about X” includes description of “X”.

As used in the present disclosure and claims, the singular forms “a”, “an” and “the” include plural forms unless the context clearly dictates otherwise.

It is understood that wherever embodiments are described herein with the term “comprising” otherwise analogous embodiments described in terms of “consisting of” and/or “consisting essentially of” are also provided. It is also understood that wherever embodiments are described herein with the phrase “consisting essentially of” otherwise analogous embodiments described in terms of “consisting of” are also provided.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

### Click-Product Binding Molecules

In some embodiments, a click product binding agent is an antibody. In some embodiments, the antibody is a recombinant antibody. In some embodiments, the antibody is a monoclonal antibody. In some embodiments, the antibody is a chimeric antibody. In some embodiments, the antibody is a humanized antibody. In some embodiments, the antibody is a human antibody. In some embodiments, the antibody is an IgA, IgD, IgE, IgG, or IgM antibody. In some embodiments, the antibody is an IgG antibody. In some embodiments, the antibody is an IgG1 antibody. In some embodiments, the antibody is an IgG2 antibody. In some embodiments, the antibody is an IgG3 antibody. In some embodiments, the antibody is an IgG4 antibody. In some embodiments, the antibody comprises a kappa light chain constant region. In some embodiments, the antibody comprises a lambda light chain constant region. In some embodiments, the antibody is an antibody fragment comprising an antigen-binding site. In some embodiments, the antibody is a diabody or a nanobody. In some embodiments, the antibody is a bispecific antibody or a multispecific antibody. In some embodiments, the antibody is a monovalent antibody. In some embodiments, the antibody is a monospecific antibody.

In some embodiments, the antibody is a bivalent antibody. In some embodiments, the antibody is a scFv. In some embodiments, the antibody is a disulfide-linked scFv (dsscFv). In some embodiments, the antibody or antibody fragment is a Fab, Fab′, F(ab′)2, Fv, scFv, (scFv)2, single chain antibody, dual variable region antibody, single variable region antibody, linear antibody, nanobody, or a V region antibody. In some embodiments, the antibody is a scFv-CH3, a scFv-Fc fusion, a scFv-HSA fusion, a scFv-PEG fusion, or a scFv-XTEN fusion.

In some embodiments, the antibody is a scFv antibody comprising a heavy chain variable region and a light chain variable region, linked by a peptide linker, e.g., a glycine and/or serine rich peptide linker. In some embodiments, the antibody is a scFv comprising a heavy chain variable region and a light chain variable region. In some embodiments of the scFv, the heavy chain variable region comprises the amino acid sequence of SEQ ID NO:1. In some embodiments of the scFv, the light chain variable region comprises the amino acid sequence of SEQ ID NO:2. In some embodiments of the scFv, the heavy chain variable region comprises the amino acid sequence of SEQ ID NO:1 and the light chain variable region comprises the amino acid sequence of SEQ ID NO:2. In some embodiments of the scFv, the dsscFv comprises the amino acid sequence of SEQ ID NOs:4-12. In some embodiments, the scFv comprises the amino acid sequence of SEQ ID NO:7.

In some embodiments, the antibody is isolated. In some embodiments, the antibody is substantially pure.

In some embodiments, a click product binding agent is a polyclonal antibody. Polyclonal antibodies can be prepared by any method known to those of skill in the art. In some embodiments, polyclonal antibodies are produced by immunizing an animal (e.g., a rabbit, rat, mouse, goat, donkey) with an antigen of interest (e.g., a purified peptide fragment, a recombinant protein, or a fusion protein) using multiple subcutaneous or intraperitoneal injections. In some embodiments, the antigen is conjugated to a carrier such as keyhole limpet hemocyanin (KLH), serum albumin, bovine thyroglobulin, or soybean trypsin inhibitor. The antigen (with or without a carrier protein) is diluted in sterile saline and usually combined with an adjuvant (e.g., Complete or Incomplete Freund's Adjuvant) to form a stable emulsion. After a period of time, polyclonal antibodies are recovered from the immunized animal (e.g., from blood or ascites). In some embodiments, the polyclonal antibodies are purified from serum or ascites according to standard methods in the art including, but not limited to, affinity chromatography, ion-exchange chromatography, gel electrophoresis, and/or dialysis.

In some embodiments, a click product binding agent is a monoclonal antibody. Monoclonal antibodies can be prepared by any method known to those of skill in the art. In some embodiments, monoclonal antibodies are prepared using hybridoma methods known to one of skill in the art. For example, using a hybridoma method, a mouse, rat, rabbit, hamster, or other appropriate host animal, is immunized as described above. In some embodiments, lymphocytes are immunized in vitro. In some embodiments, the immunizing antigen is a human protein or a fragment thereof. In some embodiments, the immunizing antigen is a cyno protein or a fragment thereof.

Following immunization, lymphocytes are isolated and fused with a suitable myeloma cell line using, for example, polyethylene glycol. The hybridoma cells are selected using specialized media as known in the art and unfused lymphocytes and myeloma cells do not survive the selection process. Hybridomas that produce monoclonal antibodies directed to a chosen antigen can be identified by a variety of methods including, but not limited to, immunoprecipitation, immunoblotting, and in vitro binding assays (e.g., flow cytometry, FACS, ELISA, SPR (e.g., Biacore), and radioimmunoassay). Once hybridoma cells that produce antibodies of the desired specificity, affinity, and/or activity are identified, the clones may be subcloned by limiting dilution or other techniques. The hybridomas can be propagated either in in vitro culture using standard methods or in vivo as ascites tumors in an animal. The monoclonal antibodies can be purified from the culture medium or ascites fluid according to standard methods in the art including, but not limited to, affinity chromatography, ion-exchange chromatography, gel electrophoresis, and dialysis.

In some embodiments, monoclonal antibodies are made using recombinant DNA techniques as known to one skilled in the art. For example, the polynucleotides encoding an antibody are isolated from mature B-cells or hybridoma cells, such as by RT-PCR using oligonucleotide primers that specifically amplify the genes encoding the heavy and light chains of the antibody and their sequence is determined using standard techniques. The isolated polynucleotides encoding the heavy and light chains are then cloned into suitable expression vectors that produce the monoclonal antibodies when transfected into host cells such as E. coli, simian COS cells, Chinese hamster ovary (CHO) cells, or myeloma cells that do not otherwise produce immunoglobulin proteins.

In some embodiments, recombinant monoclonal antibodies are isolated from phage display libraries expressing variable domains or CDRs of a desired species. Screening of phage libraries can be accomplished by various techniques known in the art.

In some embodiments, a monoclonal antibody is modified by using recombinant DNA technology to generate alternative antibodies. In some embodiments, the constant domains of the light chain and heavy chain of a mouse monoclonal antibody are replaced with the constant regions of a human antibody to generate a chimeric antibody. In some embodiments, the constant regions are truncated or removed to generate a desired antibody fragment of a monoclonal antibody. In some embodiments, site-directed or high-density mutagenesis of the variable region(s) is used to optimize specificity and/or affinity of a monoclonal antibody.

In some embodiments, a click product binding agent is a humanized antibody. Various methods for generating humanized antibodies are known in the art. In some embodiments, a humanized antibody comprises one or more amino acid residues that have been introduced into its sequence from a source that is non-human. In some embodiments, humanization is performed by substituting one or more non-human CDR sequences for the corresponding CDR sequences of a human antibody. In some embodiments, the humanized antibodies are constructed by substituting all six CDRs of a non-human antibody (e.g., a mouse antibody) for the corresponding CDRs of a human antibody.

The choice of which human heavy chain variable region and/or light chain variable region are used for generating humanized antibodies can be made based on a variety of factors and by a variety of methods known in the art. In some embodiments, the “best-fit” method is used where the sequence of the variable region of a non-human (e.g., rodent) antibody is screened against the entire library of known human variable region sequences. The human sequence that is most similar to that of the non-human (e.g., rodent) sequence is selected as the human variable region framework for the humanized antibody. In some embodiments, a particular variable region framework derived from a consensus sequence of all human antibodies of a particular subgroup of light or heavy chains is selected as the variable region framework. In some embodiments, the variable region framework sequence is derived from the consensus sequences of the most abundant human subclasses. In some embodiments, human germline genes are used as the source of the variable region framework sequences.

Other methods for humanization include, but are not limited to, (i) a method called “superhumanization” that is described as the direct transfer of CDRs to a human germline framework, (ii) a method termed Human String Content (HSC) that is based on a metric of “antibody humanness”, (iii) methods based on generation of large libraries of humanized variants (including phage, ribosomal, and yeast display libraries), and (iv) methods based on framework region shuffling.

In some embodiments, a click product binding agent is a human antibody. Human antibodies can be prepared using various techniques known in the art. In some embodiments, human antibodies are generated from immortalized human B lymphocytes immunized in vitro. In some embodiments, human antibodies are generated from lymphocytes isolated from an immunized individual. In any case, cells that produce an antibody directed against a target antigen can be generated and isolated. In some embodiments, a human antibody is selected from a phage library, where that phage library expresses human antibodies. Alternatively, phage display technology may be used to produce human antibodies and antibody fragments in vitro, from immunoglobulin variable region gene repertoires from unimmunized donors. Techniques for the generation and use of antibody phage libraries are well-known in the art. Once antibodies are identified, affinity maturation strategies known in the art, including but not limited to, chain shuffling and site-directed mutagenesis, may be employed to generate higher affinity human antibodies. In some embodiments, human antibodies are produced in transgenic mice that contain human immunoglobulin loci. Upon immunization these mice are capable of producing the full repertoire of human antibodies in the absence of endogenous immunoglobulin production.

In some embodiments, a click product binding agent is a bispecific antibody. Bispecific antibodies are capable of recognizing and binding at least two different antigens or epitopes. The different epitopes can either be within the same molecule (e.g., two epitopes on click product) or on different molecules (e.g., one epitope on click product and one epitope on a different target). In some embodiments, a bispecific antibody has enhanced potency as compared to an individual antibody or to a combination of more than one antibody. In some embodiments, a bispecific antibody has reduced toxicity as compared to an individual antibody or to a combination of more than one antibody. It is known to those of skill in the art that any therapeutic agent may have unique pharmacokinetics (PK) (e.g., circulating half-life). In some embodiments, a bispecific antibody has the ability to synchronize the PK of two active binding agents wherein the two individual binding agents have different PK profiles. In some embodiments, a bispecific antibody has the ability to concentrate the actions of two agents in a common area (e.g., tissue) in a subject. In some embodiments, a bispecific antibody has the ability to concentrate the actions of two agents to a common target (e.g., a specific cell type). In some embodiments, a bispecific antibody has the ability to target the actions of two agents to more than one biological pathway or function. In some embodiments, a bispecific antibody has the ability to target two different cells and bring them closer together.

In some embodiments, a bispecific antibody has decreased toxicity and/or side effects. In some embodiments, a bispecific antibody has decreased toxicity and/or side effects as compared to a mixture of the two individual antibodies or the antibodies as single agents. In some embodiments, a bispecific antibody has an increased therapeutic index. In some embodiments, a bispecific antibody has an increased therapeutic index as compared to a mixture of the two individual antibodies or the antibodies as single agents.

Several techniques for making bispecific antibodies are known by those skilled in the art. In some embodiments, the bispecific antibodies comprise heavy chain constant regions with modifications in the amino acids that are part of the interface between the two heavy chains. These modifications are made to enhance heterodimer formation and generally reduce or eliminate homodimer formation. In some embodiments, the bispecific antibodies are generated using a knobs-into-holes (KIH) strategy. In some embodiments, the bispecific antibodies comprise variant hinge regions incapable of forming disulfide linkages between identical heavy chains (e.g., reduce homodimer formation). In some embodiments, the bispecific antibodies comprise heavy chains with changes in amino acids that result in altered electrostatic interactions. In some embodiments, the bispecific antibodies comprise heavy chains with changes in amino acids that result in altered hydrophobic/hydrophilic interactions.

Bispecific antibodies can be intact antibodies or antibody fragments comprising antigen-binding sites.

Click product binding agents with more than two specificities are contemplated. In some embodiments, trispecific or tetraspecific antibodies are generated. click product binding agents with more than two valencies are contemplated. In some embodiments, trivalent or tetravalent antibodies are generated.

CDRs of an antibody are defined by those skilled in the art using a variety of methods/systems. These systems and/or definitions have been developed and refined over a number of years and include Kabat, Chothia, IMGT, AbM, and Contact. The Kabat definition is based on sequence variability and is commonly used. The Chothia definition is based on the location of the structural loop regions. The IMGT system is based on sequence variability and location within the structure of the variable domain. The AbM definition is a compromise between Kabat and Chothia. The Contact definition is based on analyses of the available antibody crystal structures. An Exemplary system is a combination of Kabat and Chothia. Software programs (e.g., abYsis) are available and known to those of skill in the art for analysis of antibody sequence and determination of CDRs.

The specific CDR sequences defined herein are generally based on a combination of Kabat and Chothia definitions (Exemplary system).

However, it will be understood that reference to a heavy chain variable region CDR or CDRs and/or a light chain variable region CDR or CDRs of a specific antibody will encompass all CDR definitions as known to those of skill in the art.

In some embodiments, a click product binding molecule described herein comprises the six CDRs of antibody 18, 36, 44, 51, 64, 65, 69, 72, or 83 based on the Kabat definition. In some embodiments, a click product binding molecule described herein comprises the six CDRs of antibody 18, 36, 44, 51, 64, 65, 69, 72, or 83 based on the Chothia definition. In some embodiments, a click product binding molecule described herein comprises the six CDRs of antibody 18, 36, 44, 51, 64, 65, 69, 72, or 83 based on the AbM definition. In some embodiments, a click product binding molecule described herein comprises the six CDRs of antibody 18, 36, 44, 51, 64, 65, 69, 72, or 83 based on the IMGT definition. In some embodiments, a click product binding molecule described herein comprises the six CDRs of antibody 18, 36, 44, 51, 64, 65, 69, 72, or 83 based on the contact definition. In some embodiments, a click product binding molecule described herein comprises the six CDRs of antibody 18, 36, 44, 51, 64, 65, 69, 72, or 83 based on the Exemplary definition.

In some embodiments, a click product binding agent is a click product binding molecule that comprises one, two, three, four, five, and/or six CDRs of any one of the antibodies described herein. In some embodiments, a click product binding molecule comprises (i) one, two, and/or three heavy chain variable region CDRs from an antibody described herein, and/or (ii) one, two, and/or three light chain variable region CDRs from the same antibody.

Ab51 Heavy chain variable region
(SEQ ID NO: 1)
QMQLVQSGSELKKPGASVKVSCKASGYTFTSYAMNWVRQA
PGQGLEWMGWINTNTGNPTYAQGFTGRFVFSLDTSVSTAY
LQISSLKAEDTAVYYCARFDRPRGAFDIWGQGTLVTVSS
Ab51 Light chain variable region
(SEQ ID NO: 2)
QAVLTQPSSLSASPGASASLTCTLRSGINVGTYRIYWYQQ
KPGSPPQYLLRYKSDSDKQQGSGVPSRFSGSKDASANAGI
LLISGLQSEDEADYYCMIWHSSAWVFGGGTKLTVLG

TABLE 1A
HC CDRs

| Region | Definition | Sequence Fragment | Residues | Length |
|---|---|---|---|---|
| HFR1 | Chothia | QMQLVQSGSELKKPGASVKVSCKAS----- | 1-25 | 25 |
| | AbM | QMQLVQSGSELKKPGASVKVSCKAS----- | 1-25 | 25 |
| | Kabat | QMQLVQSGSELKKPGASVKVSCKASGYTFT | 1-30 | 30 |
| | Contact | QMQLVQSGSELKKPGASVKVSCKASGYTF- | 1-29 | 29 |
| | IMGT | QMQLVQSGSELKKPGASVKVSCKAS----- | 1-25 | 25 |
| CDR-H1 | Chothia | GYTFTSY--- | 26-32 | 7 |
| | AbM | GYTFTSYAMN | 26-35 | 10 |
| | Kabat | -----SYAMN | 31-35 | 5 |
| | Contact | ----TSYAMN | 30-35 | 6 |
| | IMGT | GYTFTSYA-- | 26-33 | 8 |
| HFR2 | Chothia | AMNWVRQAPGQGLEWMGWI | 33-51 | 19 |
| | AbM | ---WVRQAPGQGLEWMG-- | 36-49 | 14 |
| | Kabat | ---WVRQAPGQGLEWMG-- | 36-49 | 14 |
| | Contact | ---WVRQAPGQGLE----- | 36-46 | 11 |
| | IMGT | -MNWVRQAPGQGLEWMGW- | 34-50 | 17 |
| CDR-H2 | Chothia | ----NTNTGN---------- | 52-57 | 6 |
| | AbM | ---WINTNTGNPT------- | 50-59 | 10 |
| | Kabat | ---WINTNTGNPTYAQGFTG | 50-66 | 17 |
| | Contact | WMGWINTNTGNPT------- | 47-59 | 13 |
| | IMGT | ----INTNTGNP-------- | 51-58 | 8 |
| HFR3 | Chothia | PTYAQGFTGRFVFSLDTSVSTAYLQISSLKAEDTAVYYCAR | 58-98 | 41 |
| | AbM | --YAQGFTGRFVFSLDTSVSTAYLQISSLKAEDTAVYYCAR | 60-98 | 39 |
| | Kabat | ---------RFVFSLDTSVSTAYLQISSLKAEDTAVYYCAR | 67-98 | 32 |
| | Contact | --YAQGFTGRFVFSLDTSVSTAYLQISSLKAEDTAVYYC-- | 60-96 | 37 |
| | IMGT | -TYAQGFTGRFVFSLDTSVSTAYLQISSLKAEDTAVYYC-- | 59-96 | 38 |
| CDR-H3 | Chothia | --FDRPRGAFDI | 99-108 | 10 |
| | AbM | --FDRPRGAFDI | 99-108 | 10 |
| | Kabat | --FDRPRGAFDI | 99-108 | 10 |
| | Contact | ARFDRPRGAFD- | 97-107 | 11 |
| | IMGT | ARFDRPRGAFDI | 97-108 | 12 |
| HFR4 | Chothia | -WGQGTLVTVSS | 109-119 | 11 |
| | AbM | -WGQGTLVTVSS | 109-119 | 11 |
| | Kabat | -WGQGTLVTVSS | 109-119 | 11 |
| | Contact | IWGQGTLVTVSS | 108-119 | 12 |
| | IMGT | -WGQGTLVTVSS | 109-119 | 11 |

TABLE 1B
LC CDRs

| Region | Definition | Sequence Fragment | Residues | Length |
|---|---|---|---|---|
| LFR1 | Chothia | QAVLTQPSSLSASPGASASLTC------ | 1-22 | 22 |
| | AbM | QAVLTQPSSLSASPGASASLTC------ | 1-22 | 22 |
| | Kabat | QAVLTQPSSLSASPGASASLTC------ | 1-22 | 22 |
| | Contact | QAVLTQPSSLSASPGASASLTCTLRSGI | 1-28 | 28 |
| | IMGT | QAVLTQPSSLSASPGASASLTCTLR--- | 1-25 | 25 |
| CDR-L1 | Chothia | TLRSGINVGTYRIY-- | 23-36 | 14 |
| | AbM | TLRSGINVGTYRIY-- | 23-36 | 14 |
| | Kabat | TLRSGINVGTYRIY-- | 23-36 | 14 |
| | Contact | ------NVGTYRIYWY | 29-38 | 10 |
| | IMGT | ---SGINVGTYR---- | 26-34 | 9 |
| LFR2 | Chothia | --WYQQKPGSPPQYLLR | 37-51 | 15 |
| | AbM | --WYQQKPGSPPQYLLR | 37-51 | 15 |
| | Kabat | --WYQQKPGSPPQYLLR | 37-51 | 15 |
| | Contact | ----QQKPGSPPQ---- | 39-47 | 9 |
| | IMGT | IYWYQQKPGSPPQYLLR | 35-51 | 17 |
| CDR-L2: | Chothia | ----YKSDSDKQQGS | 52-62 | 11 |
| | AbM | ----YKSDSDKQQGS | 52-62 | 11 |
| | Kabat | ----YKSDSDKQQGS | 52-62 | 11 |
| | Contact | YLLRYKSDSDKQQG- | 48-61 | 14 |
| | IMGT | ----YKSDSD----- | 52-57 | 6 |
| LFR3 | Chothia | -----GVPSRFSGSKDASANAGILLISGLQSEDEADYYC | 63-96 | 34 |
| | AbM | -----GVPSRFSGSKDASANAGILLISGLQSEDEADYYC | 63-96 | 34 |
| | Kabat | -----GVPSRFSGSKDASANAGILLISGLQSEDEADYYC | 63-96 | 34 |
| | Contact | ----SGVPSRFSGSKDASANAGILLISGLQSEDEADYYC | 62-96 | 35 |
| | IMGT | KQQGSGVPSRFSGSKDASANAGILLISGLQSEDEADYYC | 58-96 | 39 |
| CDR-L3 | Chothia | MIWHSSAWV | 97-105 | 9 |
| | AbM | MIWHSSAWV | 97-105 | 9 |
| | Kabat | MIWHSSAWV | 97-105 | 9 |
| | Contact | MIWHSSAW- | 97-104 | 8 |
| | IMGT | MIWHSSAWV | 97-105 | 9 |
| LFR4 | Chothia | -FGGGTKLTVLG | 106-116 | 11 |
| | AbM | -FGGGTKLTVLG | 106-116 | 11 |
| | Kabat | -FGGGTKLTVLG | 106-116 | 11 |
| | Contact | VFGGGTKLTVLG | 105-116 | 12 |
| | IMGT | -FGGGTKLTVLG | 106-116 | 11 |

In some embodiments, a click product binding agent comprises a heavy chain variable region CDR1, CDR2, and CDR3 and/or a light chain variable region CDR1, CDR2, and CDR3 from an antibody described herein. In some embodiments, a click product binding agent comprises a heavy chain variable region CDR1, CDR2, and CDR3 and a light chain variable region CDR1, CDR2, and CDR3 from an antibody described herein. In some embodiments, a click product binding agent comprises a humanized version or humanized variant of an antibody described herein.

In some embodiments, a click product binding agent comprises a heavy chain variable region CDR1, CDR2, and CDR3 and/or a light chain variable region CDR1, CDR2, and CDR3 from antibody 51, a humanized version thereof, or variants thereof. In some embodiments, a click product binding agent comprises a heavy chain variable region CDR1, a heavy chain variable region CDR2, and a heavy chain variable region CDR3 from antibody 51. In other embodiments, a click product binding agent comprises a light chain variable region CDR1, a light chain variable region CDR2, and a light chain variable region CDR3 from antibody 51. In certain embodiments, a click product binding agent comprises a heavy chain variable region CDR1, a heavy chain variable region CDR2, a heavy chain variable region CDR3, a light chain variable region CDR1, a light chain variable region CDR2, and a light chain variable region CDR3 from antibody 51. In some embodiments, a click product binding agent is a humanized version of antibody 51 (e.g., Hz51). In some embodiments, a click product binding agent is a variant of antibody 51.

In some embodiments, a click product binding agent comprises a heavy chain variable region CDR1, CDR2, and CDR3 and/or a light chain variable region CDR1, CDR2, and CDR3 from antibody 18, a humanized version thereof, or variants thereof. In some embodiments, a click product binding agent comprises a heavy chain variable region CDR1, a heavy chain variable region CDR2, and a heavy chain variable region CDR3 from antibody 18. In other embodiments, a click product binding agent comprises a light chain variable region CDR1, a light chain variable region CDR2, and a light chain variable region CDR3 from antibody 18. In certain embodiments, a click product binding agent comprises a heavy chain variable region CDR1, a heavy chain variable region CDR2, a heavy chain variable region CDR3, a light chain variable region CDR1, a light chain variable region CDR2, and a light chain variable region CDR3 from antibody 18. In some embodiments, a click product binding agent is a humanized version of antibody 18. In some embodiments, a click product binding agent is a variant of antibody 18.

In some embodiments, a click product binding agent comprises a heavy chain variable region CDR1, CDR2, and CDR3 and/or a light chain variable region CDR1, CDR2, and CDR3 from antibody 36, a humanized version thereof, or variants thereof. In some embodiments, a click product binding agent comprises a heavy chain variable region CDR1, a heavy chain variable region CDR2, and a heavy chain variable region CDR3 from antibody 36. In other embodiments, a click product binding agent comprises a light chain variable region CDR1, a light chain variable region CDR2, and a light chain variable region CDR3 from antibody 36. In certain embodiments, a click product binding agent comprises a heavy chain variable region CDR1, a heavy chain variable region CDR2, a heavy chain variable region CDR3, a light chain variable region CDR1, a light chain variable region CDR2, and a light chain variable region CDR3 from antibody 36. In some embodiments, a click product binding agent is a humanized version of antibody 36. In some embodiments, a click product binding agent is a variant of antibody 36.

In some embodiments, a click product binding agent comprises a heavy chain variable region CDR1, CDR2, and CDR3 and/or a light chain variable region CDR1, CDR2, and CDR3 from antibody 44, a humanized version thereof, or variants thereof. In some embodiments, a click product binding agent comprises a heavy chain variable region CDR1, a heavy chain variable region CDR2, and a heavy chain variable region CDR3 from antibody 44. In other embodiments, a click product binding agent comprises a light chain variable region CDR1, a light chain variable region CDR2, and a light chain variable region CDR3 from antibody 44. In certain embodiments, a click product binding agent comprises a heavy chain variable region CDR1, a heavy chain variable region CDR2, a heavy chain variable region CDR3, a light chain variable region CDR1, a light chain variable region CDR2, and a light chain variable region CDR3 from antibody 44. In some embodiments, a click product binding agent is a humanized version of antibody 5H7. In some embodiments, a click product binding agent is a variant of antibody 5H7.

In some embodiments, a click product binding agent is an antibody. In some embodiments, a variant of a click product binding molecule described herein comprises one to thirty conservative amino acid substitutions. In some embodiments, a variant of the click product binding molecule comprises one to twenty-five conservative amino acid substitutions. In some embodiments, a variant of the click product binding molecule comprises one to twenty conservative amino acid substitutions. In some embodiments, a variant of the click product binding molecule comprises one to fifteen conservative amino acid substitutions. In some embodiments, a variant of the click product binding molecule comprises one to ten conservative amino acid substitution(s). In some embodiments, a variant of the click product binding molecule comprises one to five conservative amino acid substitution(s). In some embodiments, a variant of the click product binding molecule comprises one to three conservative amino acid substitution(s). In some embodiments, the conservative amino acid substitution(s) is in a CDR of the antibody. In some embodiments, the conservative amino acid substitution(s) is not in a CDR of the antibody. In some embodiments, the conservative amino acid substitution(s) is in a framework region of the antibody.

In some embodiments, an anti-click product binding agent (e.g., antibody) comprises a heavy chain variable region comprising an amino acid sequence that has the three heavy chain variable region CDRs of antibody 51 and which has at least 75%, 80%, 85%, 90%, 95%, 96%, 97%, 98%, 99%, or 100% identity to the sequence of SEQ ID NO:1 and a light chain variable region comprising an amino acid sequence that has the three light chain variable region CDRs of antibody 51 and which has at least 75%, 80%, 85%, 90%, 95%, 96%, 97%, 98%, 99%, or 100% identity to the sequence of SEQ ID NO:2.

In some embodiments, a click product binding agent (e.g., an antibody) comprises a heavy chain variable region having at least about 80%, about 85%, about 90%, or about 95% sequence identity to SEQ ID NO:1 and/or a light chain variable region having at least about 80%, about 85%, about 90%, or about 95% sequence identity to SEQ ID NO:2. In some embodiments, a click product binding agent (e.g., an antibody) comprises a heavy chain variable region having at least about 80%, about 85%, about 90%, or about 95% sequence identity to SEQ ID NO: 1. In some embodiments, a click product binding agent (e.g., an antibody) comprises a light chain variable region having at least about 80%, about 85%, about 90%, or about 95% sequence identity to SEQ ID NO:2. In some embodiments, a click product binding agent (e.g., an antibody) comprises a heavy chain variable region having at least about 80%, about 85%, about 90%, or about 95% sequence identity to SEQ ID NO: 1 and a light chain variable region having at least about 80%, about 85%, about 90%, or about 95% sequence identity to SEQ ID NO:2. In some embodiments, a click product binding agent comprises a heavy chain variable region comprising SEQ ID NO:1 and/or a light chain variable region comprising SEQ ID NO:2. In some embodiments, a click product binding agent comprises a heavy chain variable region comprising SEQ ID NO: 1. In some embodiments, a click product binding agent comprises a light chain variable region comprising SEQ ID NO:2. In some embodiments, a click product binding agent comprises a heavy chain variable region of SEQ ID NO:1 and a light chain variable region of SEQ ID NO:2.

Provided herein are agents that compete with one or more of the antibodies described herein for binding to a click product as described herein. In some embodiments, an agent that competes with one or more of the antibodies described herein for binding to click product is an antibody. In some embodiments, an antibody binds the same epitope as one of the anti-click product antibodies described herein. In some embodiments, an antibody binds an epitope overlapping with an epitope bound by one of the anti-click product antibodies described herein. Antibodies and antigen-binding fragments that compete with, or bind to the same epitope, as the anti-click product antibodies described herein are expected to show similar functional properties.

In some embodiments, a click product binding agent described herein comprises an antibody in which at least one or more of the constant regions has been modified or deleted. In some embodiments, an antibody may comprise one or more modifications to one or more of the three heavy chain constant regions (CH1, CH2 or CH3) and/or to the light chain constant region (CL). In some embodiments, an antibody may comprise one or more modifications to the hinge region. In some embodiments, the heavy chain constant region of the modified antibody comprises at least one human constant region. In some embodiments, the heavy chain constant region of the modified antibody comprises more than one human constant region. In some embodiments, modifications to the constant region comprise additions, deletions, or substitutions of one or more amino acids in one or more regions. In some embodiments, one or more regions are partially or entirely deleted from the constant regions of a modified antibody. In some embodiments, one or more regions are partially or entirely deleted from the hinge region of a modified antibody. In some embodiments, the entire CH2 domain has been removed from an antibody. In some embodiments, a deleted constant region is replaced by a short amino acid spacer that provides some of the molecular flexibility typically imparted by the absent constant region. In some embodiments, a modified antibody comprises a CH3 domain directly fused to the hinge region of the antibody. In some embodiments, a modified antibody comprises a peptide spacer inserted between the hinge region and modified CH2 and/or CH3 domains.

It is known in the art that the constant region(s) of an antibody mediates several effector functions and these effector functions can vary depending on the isotype of the antibody. For example, binding of the C1 component of complement to the Fc region of IgG or IgM antibodies (bound to antigen) activates the complement system. Activation of complement is important in the opsonization and lysis of cell pathogens. The activation of complement also stimulates the inflammatory response and can be involved in autoimmune hypersensitivity. In addition, the Fc region of an antibody can bind a cell expressing a Fc receptor (FcR). There are a number of Fc receptors that are specific for different classes of antibody, including IgG (gamma receptors), IgE (epsilon receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell cytotoxicity or ADCC), release of inflammatory mediators, placental transfer, and control of immunoglobulin production.

In some embodiments, an antibody comprises a human native wild type or variant Fc region. The amino acid sequences of the Fc region of human IgG1, IgG2, IgG3, and IgG4 are known to those of ordinary skill in the art (e.g., a representative human IgG1 is SEQ ID NO: 13). In some cases, Fc regions with amino acid variations have been identified in native antibodies. In some embodiments, a variant Fc region is engineered with substitutions at specific amino acid positions as compared to a native Fc region (e.g., SEQ ID NO:14, SEQ ID NO:15, and SEQ ID NO:16). Exemplary native and variant IgG1 sequences are as follows:

Human native IgG1 constant region
(SEQ ID NO: 13)
ASTKGPSVFPLAPSSKSTSGGTAALGCLVKDYFPEPVTVS
WNSGALTSGVHTFPAVLQSSGLYSLSSVVTVPSSSLGTQT
YICNVNHKPSNTKVDKKVEPKSCDKTHTCPPCPAPELLGG
PSVFLFPPKPKDTLMISRTPEVTCVVVDVSHEDPEVKENW
YVDGVEVHNAKTKPREEQYNSTYRVVSVLTVLHQDWLNGK
EYKCKVSNKALPAPIEKTISKAKGQPREPQVYTLPPSREE
MTKNQVSLTCLVKGFYPSDIAVEWESNGQPENNYKTTPPV
LDSDGSFFLYSKLTVDKSRWQQGNVFSCSVMHEALHNHYT
QKSLSLSPGK
Human IgG1 constant region E233A/L235A
(SEQ ID NO: 14)
ASTKGPSVFPLAPSSKSTSGGTAALGCLVKDYFPEPVTVS
WNSGALTSGVHTFPAVLQSSGLYSLSSVVTVPSSSLGTQT
YICNVNHKPSNTKVDKKVEPKSCDKTHTCPPCPAPALAGG
PSVFLFPPKPKDTLMISRTPEVTCVVVDVSHEDPEVKFNW
YVDGVEVHNAKTKPREEQYNSTYRVVSVLTVLHQDWLNGK
EYKCKVSNKALPAPIEKTISKAKGQPREPQVYTLPPSREE
MTKNQVSLTCLVKGFYPSDIAVEWESNGQPENNYKTTPPV
LDSDGSFFLYSKLTVDKSRWQQGNVFSCSVMHEALHNHYT
QKSLSLSPGK
Human IgG1 constant region L234A/L235A
(SEQ ID NO: 15
ASTKGPSVFPLAPSSKSTSGGTAALGCLVKDYFPEPVTVS
WNSGALTSGVHTFPAVLQSSGLYSLSSVVTVPSSSLGTQT
YICNVNHKPSNTKVDKKVEPKSCDKTHTCPPCPAPEAAGG
PSVFLFPPKPKDTLMISRTPEVTCVVVDVSHEDPEVKFNW
YVDGVEVHNAKTKPREEQYNSTYRVVSVLTVLHQDWLNGK
EYKCKVSNKALPAPIEKTISKAKGQPREPQVYTLPPSREE
MTKNQVSLTCLVKGFYPSDIAVEWESNGQPENNYKTTPPV
LDSDGSFFLYSKLTVDKSRWQQGNVFSCSVMHEALHNHYT
QKSLSLSPGK
Human IgG1 constant region L234A/L235A/P329G
(SEQ ID NO: 16)
ASTKGPSVFPLAPSSKSTSGGTAALGCLVKDYFPEPVTVS
WNSGALTSGVHTFPAVLQSSGLYSLSSVVTVPSSSLGTQT
YICNVNHKPSNTKVDKKVEPKSCDKTHTCPPCPAPEAAGG
PSVFLFPPKPKDTLMISRTPEVTCVVVDVSHEDPEVKFNW
YVDGVEVHNAKTKPREEQYNSTYRVVSVLTVLHQDWLNGK
EYKCKVSNKALGAPIEKTISKAKGQPREPQVYTLPPSREE
MTKNQVSLTCLVKGFYPSDIAVEWESNGQPENNYKTTPPV
LDSDGSFFLYSKLTVDKSRWQQGNVFSCSVMHEALHNHYT
QKSLSLSPGK

In some embodiments, a modified antibody (e.g., comprising a modified Fc region) provides for altered effector functions that, in turn, affect the biological profile of the antibody. For example, in some embodiments, the deletion or inactivation (through point mutations or other means) of a constant region reduces Fc receptor binding of a modified antibody as it circulates. In some embodiments, the constant region modifications increase the serum half-life of an antibody. In some embodiments, the constant region modifications reduce the serum half-life of an antibody. In some embodiments, the constant region modifications decrease or remove ADCC and/or complement dependent cytotoxicity (CDC) of an antibody. In some embodiments, specific amino acid substitutions in a human IgG1 Fc region with corresponding IgG2 or IgG4 residues may reduce effector functions (e.g., ADCC and CDC) in a modified antibody. In some embodiments, the constant region modifications decrease or reduce ADCC and/or CDC of an antibody. In some embodiments, an antibody does not have one or more effector functions (e.g., “effectorless” antibodies). In some embodiments, an antibody has no ADCC activity and/or no CDC activity. In some embodiments, an antibody does not bind an Fc receptor and/or complement factors. In some embodiments, an antibody has no effector function(s). In some embodiments, the constant region modifications increase or enhance ADCC and/or CDC of an antibody. In some embodiments, the constant region is modified to eliminate disulfide linkages or oligosaccharide moieties. In some embodiments, the constant region is modified to add/substitute one or more amino acids to provide one or more cytotoxin, oligosaccharide, or carbohydrate attachment sites.

In some embodiments, a click product binding agent comprises an IgG1.

Modifications to the constant region of antibodies described herein may be made using well-known biochemical or molecular engineering techniques. In some embodiments, antibody variants are prepared by introducing appropriate nucleotide changes into the encoding DNA, and/or by synthesis of the desired antibody or polypeptide. Using this technique, it may be possible to disrupt the activity or effector function provided by a specific sequence or region while substantially maintaining the structure, binding activity, and other desired characteristics of the modified antibody. The present disclosure further embraces additional variants and equivalents that are substantially homologous to the recombinant, monoclonal, chimeric, humanized, and human antibodies, or antibody fragments thereof, described herein. In some embodiments, it is desirable to improve the binding affinity of the antibody. In some embodiments, it is desirable to modulate biological properties of the antibody, including but not limited to, specificity, thermostability, expression level, effector function(s), glycosylation, immunogenicity, and/or solubility. Those skilled in the art will appreciate that amino acid changes may alter post-translational processes of an antibody, such as changing the number or position of glycosylation sites or altering membrane anchoring characteristics.

Variations may be a substitution, deletion, or insertion of one or more nucleotides encoding the antibody or polypeptide that results in a change in the amino acid sequence as compared with the native antibody or polypeptide sequence. In some embodiments, amino acid substitutions are the result of replacing one amino acid with another amino acid having similar structural and/or chemical properties, such as the replacement of a leucine with a serine, for example, conservative amino acid replacements. Insertions or deletions may optionally be in the range of about 1 to 5 amino acids. In some embodiments, the substitution, deletion, or insertion includes less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the parent molecule. In some embodiments, variations in the amino acid sequence that are biologically useful and/or relevant may be determined by systematically making insertions, deletions, or substitutions in the sequence and testing the resulting variant proteins for activity as compared to the parent protein.

In some embodiments, variants may include addition of amino acid residues at the amino- and/or carboxyl-terminal end of the antibody or polypeptide. The length of additional amino acids residues may range from one residue to a hundred or more residues. In some embodiments, a variant comprises an N-terminal methionyl residue. In some embodiments, the variant comprises an additional polypeptide/protein (e.g., Fc region) to create a fusion protein. In some embodiments, a variant is engineered to be detectable and may comprise a detectable label and/or protein (e.g., a fluorescent tag or an enzyme).

In some embodiments, a cysteine residue not involved in maintaining the proper conformation of an antibody is substituted or deleted to modulate the antibody's characteristics, for example, to improve oxidative stability and/or prevent aberrant disulfide crosslinking. Conversely, in some embodiments, one or more cysteine residues are added to create disulfide bond(s) to improve stability.

In some embodiments, an antibody of the present disclosure is “deimmunized”. The deimmunization of antibodies generally consists of introducing specific amino acid mutations (e.g., substitutions, deletions, additions) that result in removal of predicted T-cell epitopes without significantly reducing the binding affinity or other desired characteristics of the antibody.

The variant antibodies or polypeptides described herein may be generated using methods known in the art, including but not limited to, site-directed mutagenesis, alanine scanning mutagenesis, and PCR mutagenesis.

In some embodiments, a click product binding agent described herein is chemically modified. In some embodiments, a click product binding agent is a click product binding molecule that has been chemically modified by glycosylation, acetylation, pegylation, phosphorylation, amidation, derivatization by known protecting/blocking groups, proteolytic cleavage, and/or linkage to a cellular ligand or other protein. Any of numerous chemical modifications may be carried out by known techniques.

The present disclosure encompasses click product binding agents built upon non-immunoglobulin backbones, wherein the agents bind the same epitope or essentially the same epitope as a click product binding molecule disclosed herein. In some embodiments, a non-immunoglobulin-based binding agent is an agent that competes with a click product binding molecule described herein in a competitive binding assay. In some embodiments, an alternative click product binding agent comprises a scaffold protein. Generally, scaffold proteins can be assigned to one of three groups based on the architecture of their backbone (1) scaffolds consisting of α-helices; (2) small scaffolds with few secondary structures or an irregular architecture of α-helices and β-sheets; and (3) scaffolds consisting of predominantly β-sheets. Scaffold proteins include, but are not limited to, anticalins, which are based upon the lipocalin scaffold; adnectins, which are based on the 10^{th} domain of human fibronectin type 3; affibodies, which are based on the B-domain in the Ig-binding region of Staphylococcus aureus protein A; darpins, which are based on ankyrin repeat domain proteins; fynomers, which are based on the SH3 domain of the human Fyn protein kinase; affitins, which are based on Sac7d from Sulfolobus acidocaldarius; affilins, which are based on human γ-B-crystallin or human ubiquitin; avimers, which are based on the A-domains of membrane receptor proteins; knottins (cysteine knot miniproteins), which are based upon a stable 30-amino acid anti-parallel β-strand protein fold; and Kunitz domain inhibitor scaffolds, which are based upon a structure that contains three disulfide bonds and three loops.

In some embodiments, a click product binding agent comprises an engineered scaffold protein comprising a heavy chain variable region CDR1, CDR2, and CDR3 and a light chain variable region CDR1, CDR2, and CDR3 from antibody 51.

Generally speaking, antigen-antibody interactions are non-covalent and reversible, formed by a combination of hydrogen bonds, hydrophobic interactions, electrostatic and van der Waals forces. When describing the strength of an antigen-antibody complex, the terms affinity and/or avidity are often used. The binding of an antibody to its antigen is a reversible process, and the affinity of the binding is typically reported as an equilibrium dissociation constant (K_{D}). K_{D} is the ratio of an antibody dissociation rate (k_{off}) (how quickly it dissociates from its antigen) to the antibody association rate (kₒₙ) (how quickly it binds to its antigen). In some embodiments, K_{D} values are determined by measuring the kₒₙ and k_{off} rates of a specific antibody/antigen interaction and then using a ratio of these values to calculate the K_{D} value. In some embodiments, K_{D} values are used to evaluate and rank the strength of individual antibody/antigen interactions. The lower the K_{D} of an antibody, the higher the affinity of the antibody for its target. In some embodiments, affinity is measured using SPR technology in a Biacore system. Avidity gives a measure of the overall strength of an antibody-antigen complex. It is dependent on three major parameters: (i) affinity of the antibody for the target, (ii) valency of both the antibody and antigen, and (iii) structural arrangement of the parts that interact.

In some embodiments, a click product binding agent (e.g., an antibody) binds click product with a K_{D} of about 1 μM or less, about 100 nM or less, about 40 nM or less, about 20 nM or less, about 10 nM or less, about 1 nM or less, about 0.1 nM or less, 50 pM or less, 10 pM or less, or 1 pM or less. In some embodiments, a click product binding agent binds click product with a K_{D} of about 20 nM or less. In some embodiments, a click product binding agent binds click product with a K_{D} of about 10 nM or less. In some embodiments, a click product binding agent binds click product with a K_{D} of about 5 nM or less. In some embodiments, a click product binding agent binds click product with a K_{D} of about 3 nM or less. In some embodiments, a click product binding agent binds click product with a K_{D} of about 2 nM or less. In some embodiments, a click product binding agent binds click product with a K_{D} of about 1 nM or less. In some embodiments, a click product binding agent binds click product with a K_{D} of about 0.5 nM or less. In some embodiments, a click product binding agent binds click product with a K_{D} of about 0.1 nM or less. In some embodiments, a click product binding agent binds click product with a K_{D} of about 50 pM or less. In some embodiments, a click product binding agent binds click product with a K_{D} of about 25 pM or less. In some embodiments, a click product binding agent binds click product with a K_{D} of about 10 pM or less. In some embodiments, a click product binding agent binds click product with a K_{D} of about 1 pM or less. In some embodiments, a click product binding agent binds click product with a K_{D} of about 0.01 nM to about 2.5 nM. In some embodiments, a click product binding agent binds click product with a K_{D} of about 0.1 nM to about 5 nM. In some embodiments, a click product binding agent binds click product with a K_{D} of about 1 nM to about 5 nM. In some embodiments, the dissociation constant of the binding agent (e.g., an antibody) to click product is the dissociation constant determined using a click product protein or a fragment thereof immobilized on a Biacore chip with the binding agent flowed over the chip. In some embodiments, the dissociation constant of the binding agent (e.g., an antibody) for click product is the dissociation constant determined using the binding agent captured on a Biacore chip with soluble click product flowed over the chip.

In some embodiments, a click product binding agent (e.g., an antibody) binds click product with a half maximal effective concentration (EC50) of about 1 pM or less, about 100 nM or less, about 40 nM or less, about 20 nM or less, about 10 nM or less, about 1 nM or less, or about 0.1 nM or less. In some embodiments, a click product binding agent binds to click product with an EC50 of about 1 pM or less, about 100 nM or less, about 40 nM or less, about 20 nM or less, about 10 nM or less, about 1 nM or less, or about 0.1 nM or less. In some embodiments, a click product binding agent binds cyno click product and/or click product with an EC50 of about 40 nM or less, about 20 nM or less, about 10 nM or less, about 1 nM or less or about 0.1 nM or less. In some embodiments, a click product binding agent binds click product with an EC50 of 0.1 nM to 3 nM, 0.1 nM to 2 nM, 0.1 nM to 1 nM, 0.5 nM to 3 nM, 0.5 nM to 2 nM, or 0.5 nM to 1 nM.

The click product binding agents (e.g., antibodies) described herein can be produced by any suitable method known in the art. Such methods range from direct protein synthesis methods to constructing a DNA sequence encoding polypeptide sequences and expressing those sequences in a suitable host. In some embodiments, a DNA sequence is constructed using recombinant technology by isolating or synthesizing a DNA sequence encoding a wild-type protein of interest. Optionally, the sequence can be mutagenized by site-specific mutagenesis to provide functional variants thereof. In some embodiments, a DNA sequence encoding a polypeptide of interest is constructed by chemical synthesis using an oligonucleotide synthesizer. Oligonucleotides can be designed based on the amino acid sequence of the desired polypeptide and selecting those codons that are favored in the host cell in which the recombinant polypeptide of interest will be produced. Standard methods can be applied to synthesize a polynucleotide sequence encoding an isolated polypeptide of interest. For example, a complete amino acid sequence can be used to construct a back-translated gene. Further, a DNA oligomer containing a nucleotide sequence coding for the particular isolated polypeptide can be synthesized. For example, several small oligonucleotides coding for portions of the desired polypeptide can be synthesized and then ligated. The individual oligonucleotides typically contain 5′ or 3′ overhangs for complementary assembly.

Once assembled (by synthesis, site-directed mutagenesis, or another method), a polynucleotide sequence encoding a particular polypeptide of interest can be inserted into an expression vector and operatively linked to an expression control sequence appropriate for expression of the protein in a desired host. Proper assembly can be confirmed by nucleotide sequencing, restriction enzyme mapping, and/or expression of a biologically active polypeptide in a suitable host. As is well-known in the art, in order to obtain high expression levels of a transfected gene in a host, the gene must be operatively linked to transcriptional and translational expression control sequences that are functional in the chosen expression host.

In some embodiments, a recombinant expression vector is used to amplify and express DNA encoding an antibody against huma click product. For example, a recombinant expression vector can be a replicable DNA construct that includes synthetic or cDNA-derived DNA fragments encoding a polypeptide chain of a click product binding agent, such as a click product binding molecule operatively linked to suitable transcriptional and/or translational regulatory elements derived from mammalian, microbial, viral or insect genes. A transcriptional unit generally comprises an assembly of (1) a genetic element or elements having a regulatory role in gene expression, for example, transcriptional promoters or enhancers, (2) a structural or coding sequence that is transcribed into mRNA and translated into protein, and (3) appropriate transcription and translation initiation and termination sequences. Regulatory elements can include an operator sequence to control transcription. The ability to replicate in a host, usually conferred by an origin of replication, and a selection gene to facilitate recognition of transformants can also be included. DNA regions are “operatively linked” when they are functionally related to each other. For example, DNA for a signal peptide (secretory leader) is operatively linked to DNA for a polypeptide if it is expressed as a precursor that participates in the secretion of the polypeptide; a promoter is operatively linked to a coding sequence if it controls the transcription of the sequence; or a ribosome binding site is operatively linked to a coding sequence if it is positioned so as to permit translation. In some embodiments, structural elements intended for use in yeast expression systems include a leader sequence enabling extracellular secretion of translated protein by a host cell. In some embodiments, in situations where recombinant protein is expressed without a leader or transport sequence, a polypeptide may include an N-terminal methionine residue. This residue can optionally be subsequently cleaved from the expressed recombinant protein to provide a final product.

The choice of an expression control sequence and an expression vector generally depends upon the choice of host. A wide variety of expression host/vector combinations can be employed. Useful expression vectors for eukaryotic hosts include, for example, vectors comprising expression control sequences from SV40, bovine papilloma virus, adenovirus, and cytomegalovirus. Useful expression vectors for bacterial hosts include known bacterial plasmids, such as plasmids from E. coli, including pCR1, pBR322, pMB9 and their derivatives, and wider host range plasmids, such as M13 and other filamentous single-stranded DNA phages.

In some embodiments, a click product binding agent (e.g., an antibody) of the present disclosure is expressed from one or more vectors. In some embodiments, a heavy chain polypeptide is expressed by one vector and a light chain polypeptide is expressed by a second vector. In some embodiments, a heavy chain polypeptide and a light chain polypeptide are expressed by one vector. In some embodiments, a vector encodes a heavy chain polypeptide of a click product binding agent described herein. In some embodiments, a vector encodes a light chain polypeptide of a click product binding agent described herein. In some embodiments, a vector encodes a heavy chain polypeptide and a light chain polypeptide of a click product binding agent described herein.

Suitable host cells for expression of a click product binding agent (e.g., an antibody) or a click product protein or fragment thereof to use as an antigen or immunogen include prokaryotes, yeast cells, insect cells, or higher eukaryotic cells under the control of appropriate promoters. Prokaryotes include gram-negative or gram-positive organisms, for example E. coli or Bacillus. Higher eukaryotic cells include established cell lines of mammalian origin as described herein. Cell-free translation systems may also be employed. Appropriate cloning and expression vectors for use with bacterial, fungal, yeast, and mammalian cellular hosts, as well as methods of protein production, including antibody production are well-known in the art.

Various mammalian culture systems may be used to express recombinant polypeptides. Expression of recombinant proteins in mammalian cells may be desirable because these proteins are generally correctly folded, appropriately modified, and biologically functional. Examples of suitable mammalian host cell lines include, but are not limited to, COS-7 (monkey kidney-derived), L-929 (murine fibroblast-derived), C127 (murine mammary tumor-derived), 3T3 (murine fibroblast-derived), CHO (Chinese hamster ovary-derived), HeLa (human cervical cancer-derived), BHK (hamster kidney fibroblast-derived), HEK-293 (human embryonic kidney-derived) cell lines and variants thereof. Mammalian expression vectors can comprise non-transcribed elements such as an origin of replication, a suitable promoter and enhancer linked to the gene to be expressed, and other 5′ or 3′ flanking non-transcribed sequences, and 5′ or 3′ non-translated sequences, such as necessary ribosome binding sites, a polyadenylation site, splice donor and acceptor sites, and transcriptional termination sequences.

Expression of recombinant proteins in insect cell culture systems (e.g., baculovirus) also offers a robust method for producing correctly folded and biologically functional proteins. Baculovirus systems for production of heterologous proteins in insect cells are well-known to those of skill in the art.

Thus, the present disclosure provides cells comprising the click product binding agents described herein. In some embodiments, the cells produce the click product binding agents described herein. In some embodiments, the cells produce an antibody designated 51, or an ScFv designated 4S. In some embodiments, the cells produce a humanized version of antibody 51. In some embodiments, the cell is a prokaryotic cell (e.g., E. coli). In some embodiments, the cell is an eukaryotic cell. In some embodiments, the cell is a mammalian cell. In some embodiments, the cell is a hybridoma cell.

Proteins produced by a host cell can be purified according to any suitable method. Standard methods include chromatography (e.g., ion exchange, affinity, and sizing column chromatography), centrifugation, differential solubility, or by any other standard technique for protein purification. Affinity tags such as hexa-histidine, maltose binding domain, influenza coat sequence, and glutathione-S-transferase can be attached to the protein to allow easy purification by passage over an appropriate affinity column. Affinity chromatography used for purifying immunoglobulins include, but are not limited to, Protein A, Protein G, and Protein L chromatography. Isolated proteins can be physically characterized using techniques known to those of skill in the art, including but not limited to, proteolysis, size exclusion chromatography (SEC), mass spectrometry (MS), nuclear magnetic resonance (NM/R), isoelectric focusing (IEF), high performance liquid chromatography (HPLC), and x-ray crystallography. The purity of isolated proteins can be determined using techniques known to those of skill in the art, including but not limited to, SDS-PAGE, SEC, capillary gel electrophoresis, IEF, and capillary isoelectric focusing (cIEF).

In some embodiments, supernatants from expression systems that secrete recombinant protein into culture media are first concentrated using a commercially available protein concentration filter, for example, an Amicon® or Millipore Pellicon® ultrafiltration unit. Following the concentration step, the concentrate can be applied to a suitable purification matrix. In some embodiments, an anion exchange resin is employed, for example, a matrix or substrate having pendant diethylaminoethyl (DEAE) groups. The matrices can be acrylamide, agarose, dextran, cellulose, or other types commonly employed in protein purification. In some embodiments, a cation exchange step is employed. Suitable cation exchangers include various insoluble matrices comprising sulfopropyl or carboxymethyl groups. In some embodiments, a hydroxyapatite media is employed, including but not limited to, ceramic hydroxyapatite (CHT). In some embodiments, one or more reverse-phase HPLC steps employing hydrophobic RP-HPLC media, e.g., silica gel having pendant methyl or other aliphatic groups, are employed to further purify a recombinant protein. In some embodiments, hydrophobic interaction chromatography (HC) is used to separate recombinant proteins based on their hydrophobicity. HIC is a useful separation technique for purifying proteins while maintaining biological activity due to the use of conditions and matrices that operate under less denaturing conditions than some other techniques. Some or all of the foregoing purification steps, in various combinations, can be employed to provide a homogeneous recombinant protein.

Click product binding agents (e.g., antibodies) of the present disclosure can be evaluated for solubility, stability, thermostability, viscosity, expression levels, expression quality, and/or purification efficiency.

In some embodiments, click product binding agents (e.g., antibodies) described herein are characterized by assays including, but not limited to, N-terminal sequencing, amino acid analysis, HPLC, mass spectrometry, ion exchange chromatography, and papain digestion.

The present disclosure also provides conjugates comprising a click product binding agent (e.g., an antibody) described herein. In some embodiments, a click product binding molecule is attached to a second molecule. In some embodiments, a click product binding molecule is conjugated to a cytotoxic agent or moiety. In some embodiments, a click product binding molecule is conjugated to a cytotoxic agent to form an ADC (antibody-drug conjugate). In some embodiments, the cytotoxic moiety is a chemotherapeutic agent including, but not limited to, methotrexate, adriamycin/doxorubicin, melphalan, mitomycin C, chlorambucil, duocarmycin, daunorubicin, pyrrolobenzodiazepines (PBDs), or other intercalating agents. In some embodiments, the cytotoxic moiety is a microtubule inhibitor including, but not limited to, auristatins, maytansinoids (e.g., DM1 and DM4), and tubulysins. In some embodiments, the cytotoxic moiety is an enzymatically active toxin of bacterial, fungal, plant, or animal origin, or fragments thereof, including, but not limited to, diphtheria A chain, non-binding active fragments of diphtheria toxin, exotoxin A chain, ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. In some embodiments, an antibody is conjugated to one or more small molecule toxins, such as calicheamicins, maytansinoids, trichothenes, and CC1065. A derivative of any one of these toxins may be used as long as the derivative retains the cytotoxic activity of the parent molecule.

Conjugates comprising a click product binding agent (e.g., an antibody) described herein may be made using any suitable method known in the art. In some embodiments, conjugates are made using a variety of bifunctional protein-coupling agents such as N-succinimidyl-3-(2-pyridyidithiol) propionate (SPDP), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene).

In some embodiments, a click product binding agent (e.g., an antibody) described herein is conjugated to a detectable substance or molecule that allows the agent to be used for diagnosis and/or detection. A detectable substance can include, but is not limited to, enzymes, such as horseradish peroxidase, alkaline phosphatase, beta-galactosidase, and acetylcholinesterase; prosthetic groups, such as biotin and flavine(s); fluorescent materials, such as, umbelliferone, fluorescein, fluorescein isothiocyanate (FITC), rhodamine, tetramethylrhodamine isothiocyanate (TRITC), dichlorotriazinylamine fluorescein, dansyl chloride, cyanine (Cy3), and phycoerythrin; bioluminescent materials, such as luciferase; radioactive materials, such as ²¹²Bi, ¹⁴C, ⁵⁷Co, ⁵¹Cr, ⁶⁷Cu, ¹⁸F, ⁶⁸Ga, ⁶⁷Ga, ¹⁵³Gd, ¹⁵⁹Gd, ⁶⁸Ge, ³H, ¹⁶⁶Ho, ¹³¹I, ¹²⁵I, ¹²³I, ¹²¹I, ¹¹⁵In, ¹¹³In, ¹¹²In, ¹¹¹In, ¹⁴⁰La, ¹⁷⁷Lu, ⁵⁴Mn, ⁹⁹Mo, ³²P, ¹⁰³Pd, ¹⁴⁹Pm, ¹⁴²Pr, ¹⁸⁶Re, ¹⁸⁸Re, ¹⁰⁵Rh, ⁹⁷Ru, ³⁵S, ⁴⁷S, ⁷⁵Se, ¹⁵³Sm, ¹¹³Sn, ¹¹⁷S, ⁸⁵Sr, ^{99m}Tc, ²⁰¹Ti, ¹³³Xe, ⁹⁰Y, ⁶⁹Yb, ¹⁷⁵Yb, ⁶⁵Zn; positron emitting metals; and magnetic metal ions.

A click product binding molecule described herein can also be conjugated to a second antibody to form an antibody heteroconjugate.

A click product binding agent (e.g., an antibody) described herein may be attached to a solid support. Such solid supports include, but are not limited to, glass, cellulose, polyacrylamide, nylon, polystyrene, polyvinyl chloride, or polypropylene. In some embodiments, an immobilized click product binding molecule is used in an immunoassay. In some embodiments, an immobilized click product binding molecule is used in purification of the target antigen (e.g., click product or cyno click product).

Polynucleotides

In some embodiments, the disclosure encompasses polynucleotides comprising polynucleotides that encode a polypeptide (e.g., a click product binding agent) described herein. The term “polynucleotides that encode a polypeptide” encompasses a polynucleotide that includes only coding sequences for the polypeptide as well as a polynucleotide that includes additional coding and/or non-coding sequences. The polynucleotides of the disclosure can be in the form of RNA or in the form of DNA. DNA includes cDNA, genomic DNA, and synthetic DNA; and can be double-stranded or single-stranded, and if single stranded can be the coding strand or non-coding (anti-sense) strand.

In some embodiments, a polynucleotide comprises a polynucleotide (e.g., a nucleotide sequence) encoding a heavy chain of a click product binding agent (e.g., antibody) described herein. In some embodiments, a polynucleotide comprises a polynucleotide encoding a light chain of a click product binding agent (e.g., antibody) described herein. In some embodiments, a polynucleotide comprises a polynucleotide encoding a heavy chain of a click product binding agent (e.g., antibody) described herein and a polynucleotide encoding a light chain of the click product binding agent (e.g., antibody).

In some embodiments, the polynucleotide comprises a polynucleotide (e.g., a nucleotide sequence) encoding a polypeptide comprising an amino acid sequence described herein. In some embodiments, the polynucleotide comprises a polynucleotide (e.g., a nucleotide sequence) encoding a polypeptide comprising an amino acid sequence of SEQ ID NO:1 or 2. In some embodiments, the polynucleotide comprises a polynucleotide encoding a polypeptide comprising an amino acid sequence of SEQ ID NO:7.

The present disclosure also provides variants of the polynucleotides described herein, wherein the variant encodes, for example, fragments, analogs, and/or derivatives of a polypeptide. In some embodiments, the present disclosure provides a polynucleotide comprising a polynucleotide having a nucleotide sequence at least about 80% identical, at least about 85% identical, at least about 90% identical, at least about 95% identical, and in some embodiments, at least about 96%, 97%, 98% or 99% identical to a polynucleotide encoding a polypeptide described herein.

In some embodiments, a polynucleotide comprises a polynucleotide having a nucleotide sequence at least about 80% identical, at least about 85% identical, at least about 90% identical, at least about 95% identical, and in some embodiments, at least about 96%, 97%, 98%, or 99% identical to a polynucleotide encoding an amino acid sequence selected from the group consisting of SEQ ID NOs:4-12, e.g., SEQ ID NO:7. Also provided is a polynucleotide that comprises a polynucleotide that hybridizes to a polynucleotide encoding an amino acid sequence selected from the group consisting of: SEQ ID NOs:4-12, e.g., SEQ ID NO:7. In some embodiments, the hybridization is under conditions of high stringency as is known to those skilled in the art.

As used herein, the phrase “a polynucleotide having a nucleotide sequence at least, for example, 95% identical to a reference nucleotide sequence” is intended to mean that the nucleotide sequence of the polynucleotide is identical to the reference sequence except that the polynucleotide sequence can include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence can be deleted or substituted with another nucleotide, or a number of nucleotides up to 5% of the total nucleotides in the reference sequence can be inserted into the reference sequence. These mutations of the reference sequence can occur at the 5′ or 3′ terminal positions of the reference nucleotide sequence or anywhere between those terminal positions, interspersed either individually among nucleotides in the reference sequence or in one or more contiguous groups within the reference sequence.

The polynucleotide variants can contain alterations in the coding regions, non-coding regions, or both. In some embodiments, a polynucleotide variant contains alterations that produce silent substitutions, additions, or deletions, but does not alter the properties or activities of the encoded polypeptide. In some embodiments, a polynucleotide variant comprises silent substitutions that results in no change to the amino acid sequence of the polypeptide (due to the degeneracy of the genetic code). In some embodiments, a polynucleotide variant comprises one or more mutated codons comprising one or more (e.g., 1, 2, or 3) substitutions to the codon that change the amino acid encoded by that codon. Methods for introducing one or more substitutions into a codon are known in the art, including but not limited to, PCR mutagenesis and site-directed mutagenesis. Polynucleotide variants can be produced for a variety of reasons, for example, to optimize codon expression for a particular host (e.g., change codons in the human mRNA to those preferred by a bacterial host such as E. coli). In some embodiments, a polynucleotide variant comprises at least one silent mutation in a non-coding or a coding region of the sequence.

In some embodiments, a polynucleotide comprises the coding sequence for a polypeptide (e.g., an antibody) fused in the same reading frame to a polynucleotide that aids in expression and secretion of a polypeptide from a host cell (e.g., a leader sequence that functions as a secretory sequence for controlling transport of a polypeptide). The polypeptide can have the leader sequence cleaved by the host cell to form a “mature” form of the polypeptide.

In some embodiments, a polynucleotide comprises the coding sequence for a polypeptide (e.g., an antibody) fused in the same reading frame to a marker or tag sequence. For example, in some embodiments, a marker sequence is a hexa-histidine (SEQ ID NO:20) tag (HIS-tag) that allows for efficient purification of the polypeptide fused to the marker. In some embodiments, a marker sequence is a hemagglutinin (HA) tag derived from the influenza hemagglutinin protein when a mammalian host (e.g., COS-7 cells) is used. In some embodiments, the marker sequence is a FLAG™ tag. In some embodiments, a marker is used in conjunction with other markers or tags.

In some embodiments, the polynucleotides are isolated. In some embodiments, the polynucleotides are substantially pure.

Vectors and cells comprising the polynucleotides described herein are also provided. In some embodiments, an expression vector comprises a polynucleotide molecule encoding a click product binding agent (e.g., an antibody) described herein. In some embodiments, an expression vector comprises a polynucleotide molecule encoding a polypeptide that is part of a click product binding agent described herein. In some embodiments, a host cell comprises an expression vector comprising the polynucleotide molecule encoding a click product binding agent described herein. In some embodiments, a host cell comprises an expression vector comprising the polynucleotide molecule encoding a polypeptide that is part of a click product binding agent described herein. In some embodiments, a host cell comprises a polynucleotide molecule encoding a click product binding agent described herein. In some embodiments, a cell comprises one or more polynucleotides encoding a click product binding agent described herein. In some embodiments, a cell comprises a single polynucleotide encoding a click product binding agent described herein. In some embodiments, a cell comprises a first polynucleotide encoding a heavy chain of a click product binding agent described herein and a second polynucleotide encoding a light chain of a click product binding agent described herein. In some embodiments, a cell comprises a polynucleotide encoding a heavy chain and a light chain of a click product binding agent described herein. In some embodiments, a cell comprises one or more vectors encoding a click product binding agent described herein. In some embodiments, a cell comprises a vector encoding a click product binding agent described herein. In some embodiments, a cell comprises a first vector encoding a heavy chain of a click product binding agent described herein and a second vector encoding a light chain of a click product binding agent described herein. In some embodiments, a cell comprises a single vector encoding a heavy chain and a light chain of a click product binding agent described herein.

Methods of Making Binding Agents

Described herein are methods for making the click product binding agents (e.g., antibodies) described herein. In some embodiments, a method comprises providing a cell comprising a heavy chain and/or light chain of a click product binding agent (e.g., an antibody), incubating the cell under conditions that permit the expression of the binding agent, and isolating the binding agent. In certain embodiments, the cell comprises one or more vectors encoding the heavy chain and the light chain of a click product binding molecule described herein. In some embodiments, a cell comprises a first vector encoding the heavy chain of a click product binding molecule described herein and a second vector encoding the light chain a click product binding molecule described herein. In other embodiments, a cell comprises a vector encoding the heavy chain and the light chain of a click product binding molecule described herein. In certain embodiments, a cell comprises one or more polynucleotides encoding the heavy chain and the light chain of a click product binding molecule described herein. In some embodiments, a cell comprises a first polynucleotide encoding the heavy chain of a click product binding molecule described herein and a second polynucleotide encoding the light chain of a click product binding molecule described herein. In other embodiments, a cell comprises a polynucleotide encoding the heavy chain and the light chain of a click product binding molecule described herein. In some embodiments, the method comprises purifying the antibody. In certain embodiments, the cell is a CHO cell. In some embodiments, the cell is a 293 cell. In certain embodiments, the cell is a bacterial cell (e.g., E. coli).

Compositions Comprising Binding Agents

The present disclosure provides compositions comprising a click product binding agent described herein. The present disclosure also provides pharmaceutical compositions comprising a click product binding agent described herein and a pharmaceutically acceptable vehicle.

Formulations are prepared for storage and/or use by combining a click product binding agent (e.g., antibody) of the present disclosure with a pharmaceutically acceptable vehicle (e.g., a carrier or excipient). Those of skill in the art generally consider pharmaceutically acceptable carriers, excipients, and/or stabilizers to be inactive ingredients of a formulation or pharmaceutical composition.

Suitable pharmaceutically acceptable vehicles include, but are not limited to, nontoxic buffers such as phosphate, citrate, and other organic acids; salts such as sodium chloride; antioxidants including ascorbic acid and methionine; preservatives such as octadecyldimethylbenzyl ammonium chloride, hexamethonium chloride, benzalkonium chloride, benzethonium chloride, phenol, butyl or benzyl alcohol, alkyl parabens, such as methyl or propyl paraben, catechol, resorcinol, cyclohexanol, 3-pentanol, and m-cresol; low molecular weight polypeptides (e.g., less than about 10 amino acid residues); proteins such as serum albumin, gelatin, or immunoglobulins; hydrophilic polymers such as polyvinylpyrrolidone; amino acids such as glycine, glutamine, asparagine, histidine, arginine, or lysine; carbohydrates such as monosaccharides, disaccharides, glucose, mannose, or dextrins; chelating agents such as EDTA; sugars such as sucrose, mannitol, trehalose or sorbitol; salt-forming counter-ions such as sodium; metal complexes such as Zn-protein complexes; and non-ionic surfactants such as TWEEN or polyethylene glycol (PEG). (Remington: The Science and Practice of Pharmacy, 22^{nd} Edition, 2012, Pharmaceutical Press, London.). In some embodiments, the formulation is in the form of an aqueous solution. In some embodiments, the formulation is lyophilized or in an alternative dried form.

The therapeutic formulation can be in unit dosage form. Such formulations include tablets, pills, capsules, powders, granules, solutions or suspensions in water or non-aqueous media, or suppositories. In solid compositions such as tablets the principal active ingredient is mixed with a pharmaceutical carrier. Conventional tableting ingredients include corn starch, lactose, sucrose, sorbitol, talc, stearic acid, magnesium stearate, dicalcium phosphate or gums, and diluents (e.g., water). These can be used to form a solid preformulation composition containing a homogeneous mixture of a compound of the present disclosure, or a non-toxic pharmaceutically acceptable salt thereof. The solid preformulation composition is then subdivided into unit dosage forms of a type described above. The tablets, pills, etc. of the formulation or composition can be coated or otherwise compounded to provide a dosage form affording the advantage of prolonged action. For example, the tablet or pill can comprise an inner composition covered by an outer component. Furthermore, the two components can be separated by an enteric layer that serves to resist disintegration and permits the inner component to pass intact through the stomach or to be delayed in release. A variety of materials can be used for such enteric layers or coatings, such materials include a number of polymeric acids and mixtures of polymeric acids with such materials as shellac, cetyl alcohol, and cellulose acetate.

The binding agents of the present disclosure may be formulated in any suitable form for delivery to a target cell/tissue. In some embodiments, a click product binding agent can be formulated as a liposome, microparticle, microcapsule, albumin microsphere, microemulsion, nano-particle, nanocapsule, or macroemulsion. In some embodiments, the pharmaceutical formulation includes a click product binding agent of the present disclosure complexed with liposomes. Methods to produce liposomes are known to those of skill in the art. For example, some liposomes can be generated by reverse phase evaporation with a lipid composition comprising phosphatidylcholine, cholesterol, and PEG-derivatized phosphatidylethanolamine (PEG-PE).

In some embodiments, a click product binding agent is formulated as a sustained-release preparation. Suitable examples of sustained-release preparations include semi-permeable matrices of solid hydrophobic polymers containing an agent, where the matrices are in the form of shaped articles (e.g., films or microcapsules). Sustained-release matrices include but are not limited to polyesters, hydrogels such as poly(2-hydroxyethyl-methacrylate) or poly(vinyl alcohol), polylactides, copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

The pharmaceutical compositions or formulations of the present disclosure can be administered in any number of ways for either local or systemic treatment. In some embodiments, administration is topical by epidermal or transdermal patches, ointments, lotions, creams, gels, drops, suppositories, sprays, liquids and powders. In some embodiments, administration is pulmonary by inhalation or insufflation of powders or aerosols, including by nebulizer, intratracheal, and intranasal. In some embodiments, administration is oral. In some embodiments, administration is parenteral including intravenous, intraarterial, intratumoral, subcutaneous, intraperitoneal, intramuscular (e.g., injection or infusion), or intracranial (e.g., intrathecal or intraventricular). In some embodiments, administration is by intravenous injection or intravenous infusion.

Various delivery systems are known and can be used to administer a click product binding agent described herein. In some embodiments, a click product binding agent or a composition described herein is delivered in a controlled release or sustained release system. In some embodiments, a pump is used to achieve controlled or sustained release. In some embodiments, polymeric materials are used to achieve controlled or sustained release of the click product binding agent described herein. Examples of polymers used in sustained release formulations include, but are not limited to, poly 2-hydroxy ethyl methacrylate, polymethyl methacrylate, polyacrylic acid, polyethylene-co-vinyl acetate, polymethacrylic acid, polyglycolides (PLG), polyanhydrides, poly N-vinyl pyrrolidone, polyvinyl alcohol (PVA), polyacrylamide, polyethylene glycol (PEG), polylactides (PLA), polylactide-co-glycolides (PLGA), and polyorthoesters. Any polymer used in a sustained release formulation should be inert, free of leachable impurities, stable on storage, sterile, and biodegradable.

Additional delivery systems can be used to administer a click product binding agent described herein including, but not limited to, injectable drug delivery devices and osmotic pumps. Injectable drug delivery devices include, for example, hand-held devices (e.g., autoinjectors) or wearable devices. Different types of osmotic pump systems may include single compartment systems, dual compartment systems, and multiple compartment systems.

### Assays and or Kits Comprising Click Product Binding Agents

In some embodiments, the click product binding agents (e.g., anti-click product antibodies) described herein are useful for detecting the presence of click product in a biological sample. The term “detecting” as used herein encompasses quantitative or qualitative detection. In some embodiments, a biological sample comprises a cell, tissue, blood, or other bodily fluid.

In some embodiments, a method of detecting the presence of click product binding agent in a biological sample comprises contacting the biological sample with a click product binding agent under conditions permissive for binding of the click product binding molecule to click product, and detecting whether a complex is formed between the click product binding agent and a molecule comprising a click product. The methods may include assays known by those of skill in the art, such as Western blot analyses, radioimmunoassays, ELISAs, “sandwich” immunoassays, SPR (e.g., Biacore), immunoprecipitation assays, fluorescent immunoassays, protein A immunoassays, and immunohistochemistry (IHC).

In some embodiments, the click product binding agent is tagged with a detectable label. Useful detectable labels include, but are not limited to, fluorescent molecules, chemiluminescent molecules, bioluminescent molecules, enzymes, and radioisotopes. The present disclosure provides kits that comprise the click product binding agents described herein. In some embodiments, a kit is used to perform the methods described herein. In some embodiments, a kit comprises at least one purified click product binding agent (e.g., an antibody) in one or more containers. In some embodiments, the kits contain all of the components necessary and/or sufficient to perform a detection assay, including all controls, directions for performing assays, and any necessary software for analysis and presentation of results. One skilled in the art will readily recognize that the disclosed click product binding agents of the present disclosure can be readily incorporated into one of the established kit formats that are well known in the art.

### Applications of Click-Product Binding Molecules

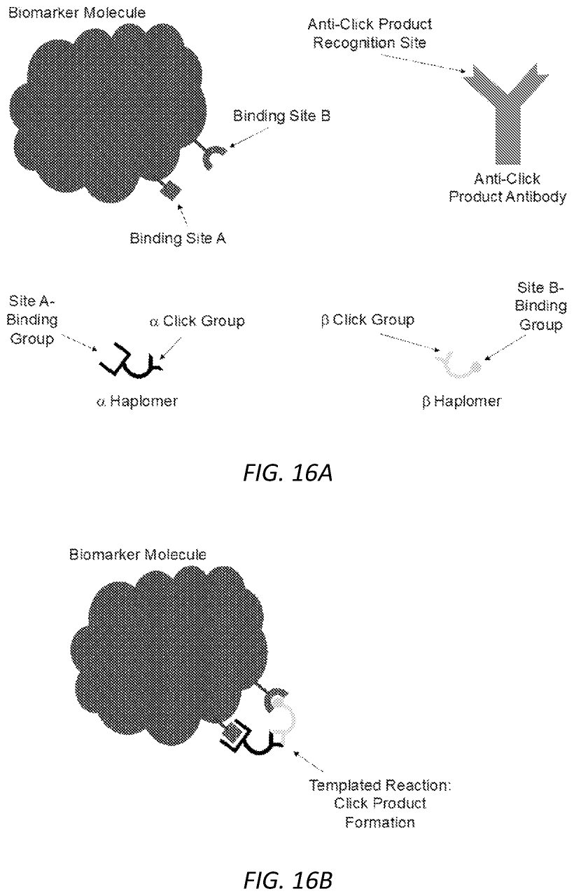

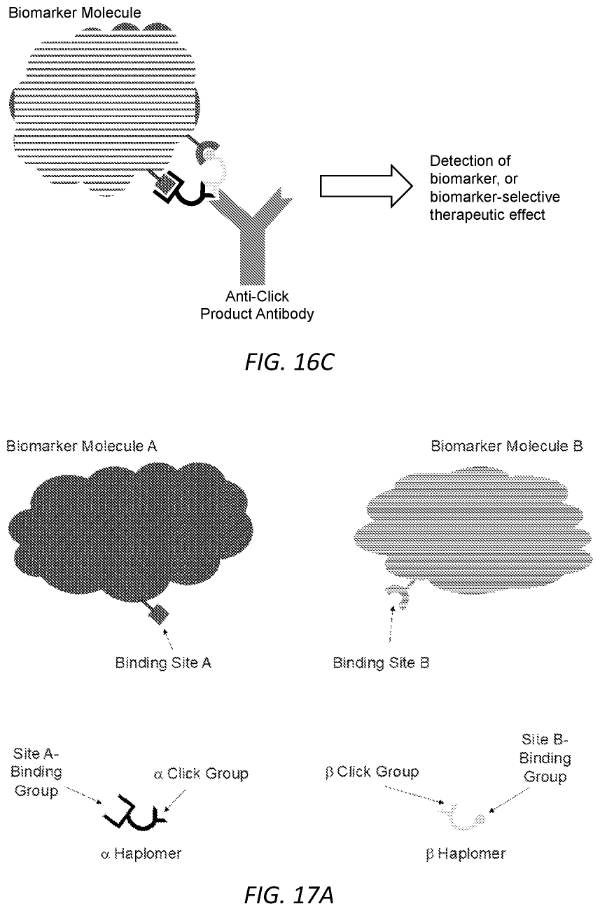

The present binding molecules have a number of uses, including binding/detecting click products formed by templated assembly. For example, haplomers (e.g., as described in WO2014197547) can be utilized to selectively create click products in situ in the presence of specific biomolecule targets (See, e.g., FIGS. 16A-C). The click-product binding molecules can then be utilized to detect the presence of the target, alter the target's activity, or direct therapy to cells harboring the target.

Thus provided herein are methods using the click-product binding molecules to detect, sort, or isolate subpopulations of cells containing a target molecule. Thus, the click-product binding molecules can be used to diagnose disease using Haplomers together with anti-click-product antibody to detect presence of a disease biomarker; to treat disease using Haplomers together with anti-click-product antibody to target a biomolecule in diseased cells and thereby either induce or inhibit the activity of the target biomolecule; to treat disease using Haplomers together with anti-click-product antibody to target a biomolecule in diseased cells and thereby inducing destruction of the diseased cells by immune signaling (ADCC) or direct toxicity if the antibody is conjugated to a toxic drug (antibody drug conjugate); or to treat disease by incorporating the sequence of the anti-click-product antibody into a CAR-T molecule for cell-based therapies. For example, this could be used to target tumors with defined targetable surface markers (including but not limited to leukemias, lymphomas, breast cancer, colon cancer, melanoma), or to delete self-reactive cells in autoimmune diseases where a B cell/plasma cell specific population produce autoimmune antibodies.

These methods have a number of advantages. Unlike most existing antibody-based approaches to achieve the above aims, the antibody is universal in this case: a new antibody does not have to be developed for each new target of interest. New Haplomers must be developed for each new target, but relative to antibodies these are much cheaper and easier to synthesize, and screening is simpler and faster. In addition, since the antibody is directed against an unnatural (non-biochemical) click structure, the likelihood of cross-reactivity with non-target biomolecules is expected to be reduced compared with typical biomolecule-directed antibodies. Also, since antibody binding to target is mediated via Haplomers, binding can be controlled and titred to desired levels by controlling Haplomer concentration. Binding can also be reversed in the event of adverse reactions by administration of small, inert click-product competitor.

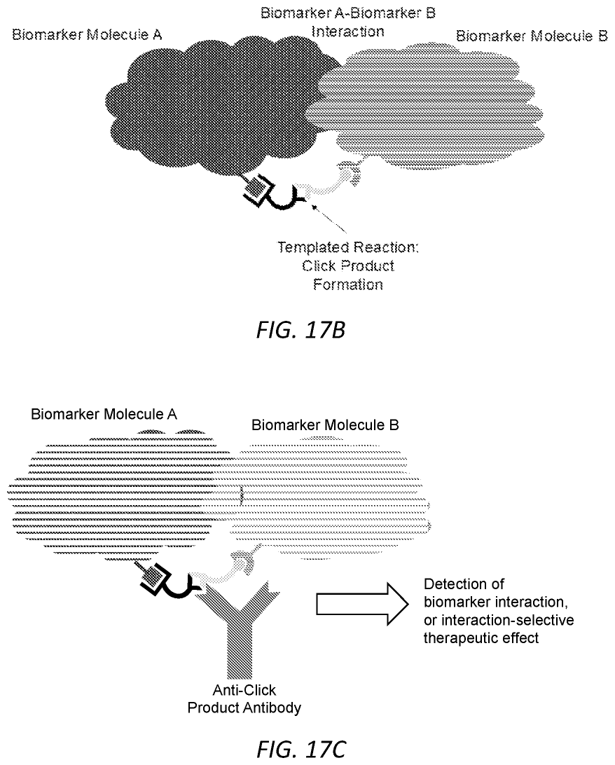

In addition, the present compositions and methods can be used for detecting interactions between two target molecules, and/or selectively binding/marking cells in which specific interactions have occurred. In some embodiments, the methods are similar to those described immediately above, except that the Haplomers (or other click-functionalized target-binding molecules) are designed to bind two or more different molecules that interact in diseased cells, rather than a single molecule (see, e.g., FIGS. 17A-C). These two-target methods can be used as a detection method for Proximity Ligation Assays or protein-protein interaction screening. In addition, the two-target methods can be used for the same diagnostic and therapeutic applications described above, except that the Haplomers are designed to bind two or more different molecules that interact in diseased cells, potentially providing increased specificity. These two-target methods have additional advantages over the above, as this approach allows targeting of interactions between different biomolecules, opening up a new class of potential drug targets.

These methods have similar diagnostic and therapeutic applications as described above, except that the click products are formed by metabolic labeling processes rather than Haplomers. In addition, these methods have the additional advantage of utilizing more flexible, robust, and selective chemistry than other methods of detecting metabolically labeled cells.

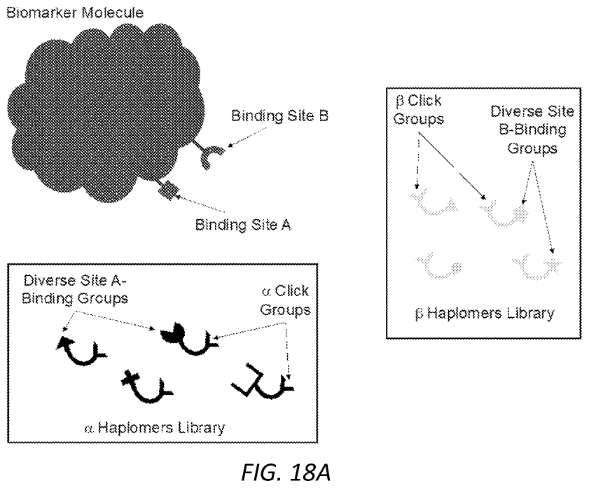

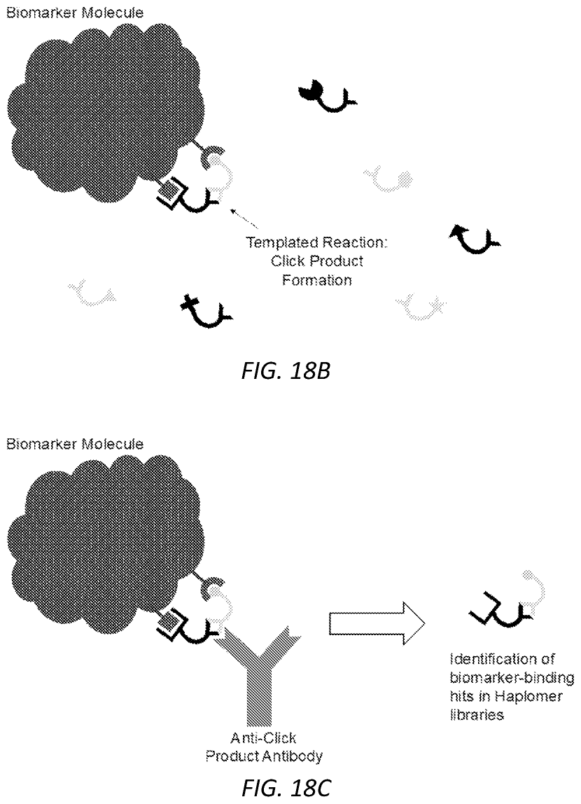

Additionally, the present methods and compositions can be used for screening drug candidates formed by kinetic target-guided synthesis. For example, click chemistry has found significant utility in drug discovery by facilitating target-guided synthesis, in which candidate fragments are functionalized with click chemistry groups and then screened against a target biomolecule. The target molecule catalyzes the formation of click products from two or more fragments that bind in proximity on the target. The click-product binding molecules can be used to detect or isolate the products thus formed. See FIGS. 18A-C.

See also Meghani et al., Drug Discov Today. 2017 November; 22(11):1604-1619; Jiang et al., Expert Opin Drug Discov. 2019 August; 14(8):779-789; Xu and Jones, Pharm Pat Anal. 2015; 4(2):109-19; Kim and Koo, Chem Sci. 2019 Aug. 16; 10(34):7835-7851. and Begley et al., Methods Enzymol. 2011; 493: 533-556.

Further, the click-product binding molecules can serve as a universal antibody effector for target-binding molecules formed in kinetic target-guided screening. For example, anti-click-product antibodies together with binding fragment pairs isolated from target-guided synthesis could further be utilized to diagnose or treat disease associated with the target biomolecule. Since the screening is rapid and the same antibody can be utilized regardless of the target, this approach could facilitate rapid screening and deployment of antibody-based therapies against newly emerging diseases, such as novel viruses or bacteria. This approach essentially combines the above Application 4 (as the first step) and Application 1 (as the second step).

### EXAMPLES

### Example 1. Design of Peptide Flanking Sequences to Click Groups

Peptide sequences flanking a clicked product between azide and DBCO moieties should be of low immunogenicity in isolation, but confer favorable recognition properties for an antibody after formation of the clicked product.

The general design premise, based on knowledge of typical lengths of contiguous peptide sequences recognized by antibodies (generally about 8-10 amino acid residues; Andresen and Bier, Methods Mol Biol. 2009; 509:123-34). was a click peptide of structure (1):

[A]-<azide-DBCO>-[B] (1),
where [A] and [B] represent distinct tetrapeptide sequences. Thus, the click product of peptides [A]-azide and DBCO-[B] produces structure (1), which is within the general size range encompassed by antibody variable region antigen binding sites.

Existing knowledge of large numbers of antibody epitopes indicates that their sequences are strongly non-random, with the implication that certain sequences are poorly compatible with being recognized by antibody complementarity-determining regions (CDRs). Therefore, de novo design of peptide sequences flanking a click-product core should accommodate such information. A rational basis for such peptide design is thus to favor sequences known to occur within antibody epitopes, or which conform to general epitope structural patterns such as probability of protein surface exposure. Also, residues prone to oxidative reactions or present in known epitopes at low frequencies should be excluded (C, M, W). With the remaining 17-member amino acid alphabet, random 17-mer strings were tested for predicted immunogenicity patterns for 8-mers (with the public online platform imed.med.ucm.es/Tools/antigenic.pl).

Although it was desirable to identify candidate novel click-product flanking sequences that were well-compatible with principles of antibody recognition, at the same time it was important to minimize the chances that a selected anti-click peptide antibody would cross-react with endogenous proteins of human origin. Also, peptide sequences corresponding to all known biologically active mediators should likewise be removed from further consideration.

As a means of approaching the ideal of anti-click antibody non-reactivity towards the human proteome (that is, antibody bio-orthogonality), peptide strings corresponding to candidate 8-mers from the above immunogenic pattern screening were given central YY or FF residues as a surrogate for the hydrophobic azide-DBCO product. Thus, an 8-mer peptide would be rendered as (N-terminal tetramer) YY/FF (C-terminal tetramer). These 10-mer sequences were then used for protein-BLAST searches to find the lowest levels of database matching, especially for the human proteome. In addition, candidates were checked to ensure absence of matching sites for known mediators, as noted above.

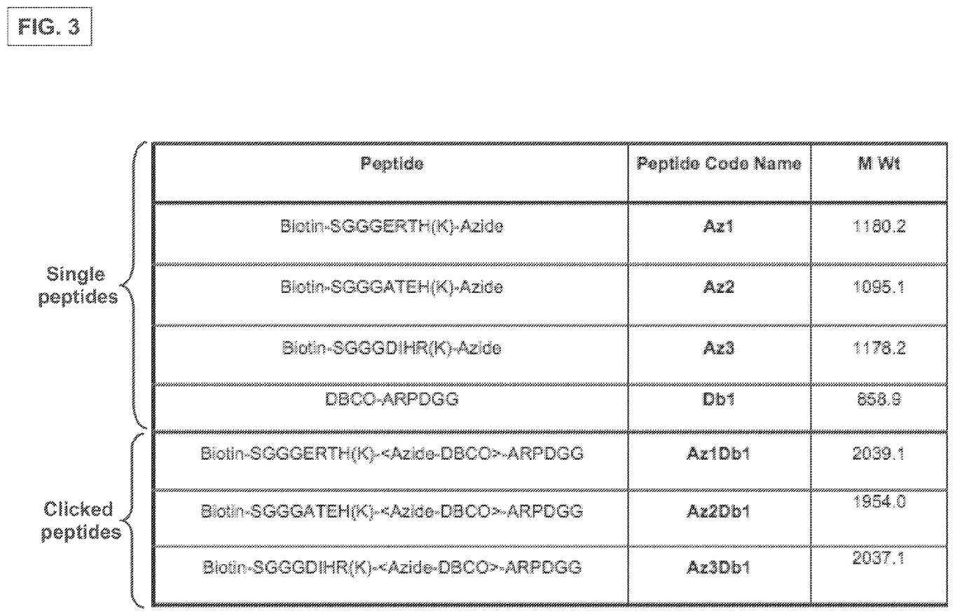

After all preliminary in silico screening steps, the final set of peptides designed for scFv screening is shown in FIG. 3. The final structures included three octameric peptides (Az1, Az2, and Az3) with N-terminal biotins for streptavidin-based surface immobilization, each with C-terminal azide moieties to be chemically appended via NHS reaction with the epsilon-amino groups of added C-terminal lysines. Each azide-peptide contained a 4-residue serine-glycine linker between the biotin group and the core peptides relevant to the planned scFv phage selection process, in order to promote free access to the click products and the flanking core peptides when surface-immobilized with solid-phase streptavidin.

In addition, a single hexameric peptide with an N-terminally-conjugated DBCO group (via the N-terminal amino; db1) was designed such that it could react with each azide-peptide, to produce Az1db1, Az2db1, and Az3db1 (FIG. 3).

### Example 2. Generation and Testing of Peptide Click Products

Peptides were synthesized by BioSynthesis Inc in accordance with the above designs, and reconstituted in aqueous solution to 10 mM. For click reactions, peptides were combined at high concentrations in order to promote mutual reactivity in the absence of template. A typical reaction used 4.5 mM concentrations of both azide- and DBCO-peptides in 50 mM sodium phosphate buffer pH 7.0/100 mM NaCl, for 24 hr at room temperature.

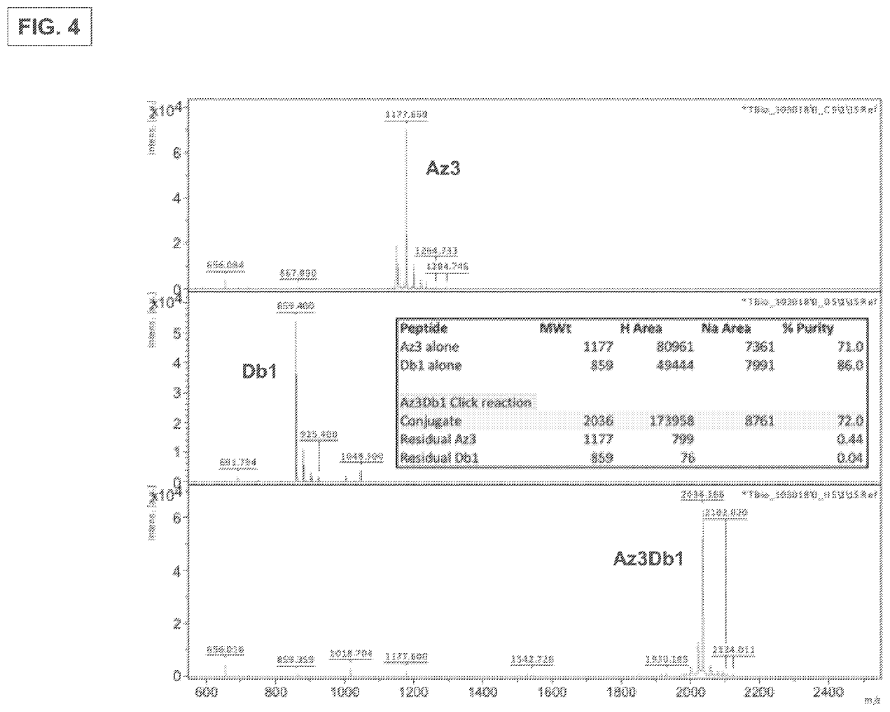

Mass spectrometry (MS) was used for analysis of extent of conversion of the individual peptides into clicked products in each case. A typical MS profile is shown in FIG. 4, for the peptides Az3, db1, and their clicked product Az3db1. Conversion rates of ≥70% were considered useful for selection purposes, given that counter-selections with unreacted peptides were an inherent part of the designed scFv selection process.

Unreacted and clicked peptides were evaluated for binding by samples of human and mouse sera. Only very low-titer responses were seen, likely attributable to binding by low-affinity polyspecific antibodies. This was evident since absorption of sera with varying click peptides diminished responses not only to the absorbing peptide, but the other click peptides as well. In view of this, these weak low-specificity events were judged to be unlikely to affect the future deployment of a selected anti-click antibody with the chosen peptide sequences (FIG. 3).

### Example 3. Isolation of Candidate Click-Product Binders from scFv Phage Display Libraries

Screening of scFv libraries was performed using surface display of scFv molecules as fusions with the Gene III protein of filamentous M13 bacteriophage, with a phagemid system that has been widely exploited. Initially a mixture of three click peptides (Az1db1, Az2db1 and Az3db1; FIG. 3) was used for the screenings, in order to streamline the isolation of binders towards any one of these targets, with a specific proprietary human phage display library. Four successive rounds of positive selection on streptavidin-immobilized target peptide were performed with additional accompanying rounds of negative selection to remove scFvs that had significant binding affinity towards any of the single unclicked peptide precursors of the clicked desired targets (FIG. 3). Such negative selections involved both panning of enriched selected sublibraries with immobilized single-component peptides, and addition of the latter peptides as competitors during rounds of positive selection. However, although candidate binders were obtained from this library by these successive steps, none showed strong signals in primary screening towards click targets in ELISA assays. Sequence analysis of these candidates subsequently demonstrated that most were deletion mutants of all or part of Gene III, proving that only spurious non-specific binding could result with these phage.

In view of these findings, a second proprietary human phage display library HuScL-6 (capacity 10¹¹ clones) was used for screening a single click target only (Az3db1) in order to focus binding selection on a specific target. Negative selections were performed here as for the first library, but with Az3 and db1 single-component peptides only. From the HuScL-6 library, a set of nine independent scFv phage clone candidates with strong ELISA signals towards the Az3db1 target, but not to Az3 or db1 alone, were isolated.

Sequences of Candidate Anti-Click Product scFv Molecules

Following determination of the encoded scFv DNA sequences, the corresponding protein sequences for the nine scFv candidates are provided as below, for the segment order V_{H}—(exemplary serine-glycine linker comprising GGGGSGGGGSGGGGS (SEQ ID NO:3); lower case and bold)—V_{L}. Complementarity-determining regions (CDRs) are boxed. Each scFv has been assigned a number (scFv-18, 36, 44, 51, 64, 65, 69, 72, and 83); each member of the subset of five that yielded sufficient expressed protein for characterization has been given a subcode 1S-5S for convenience. (1S=scFv-18; 2S=scFv-36; 3S=scFv-44; 4S=scFv-51; 5S=scFv-65).

18 (scFv-1S)
(SEQ ID NO: 4)

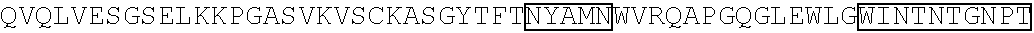

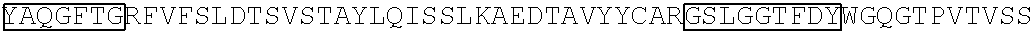

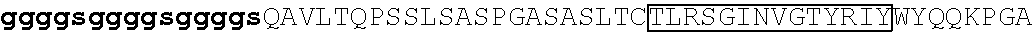

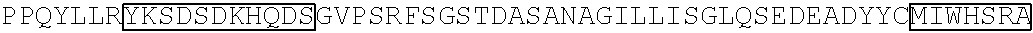

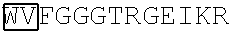

36 (szcFv-2S)
(SEQ ID NO: 5)

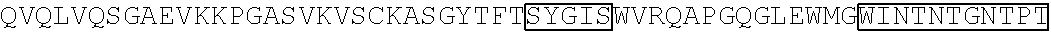

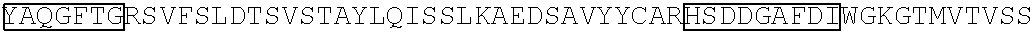

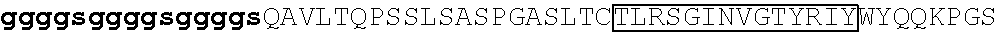

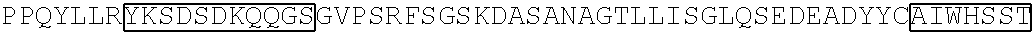

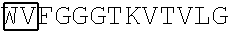

44 (scFv-3S)
(SEQ ID NO: 6)

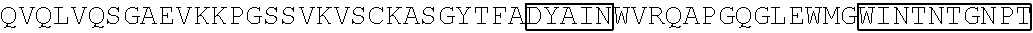

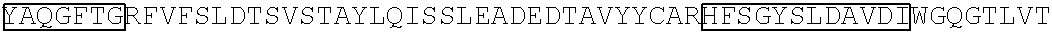

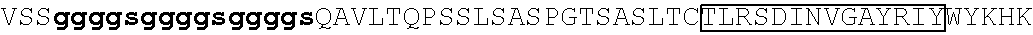

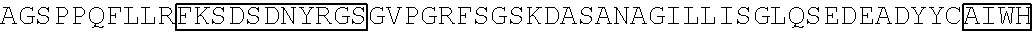

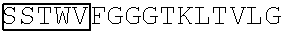

51 (scFv-4S)
(SEQ ID NO: 7)

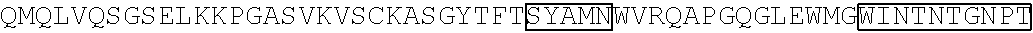

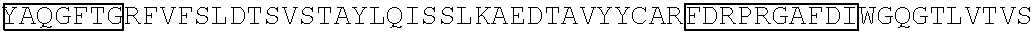

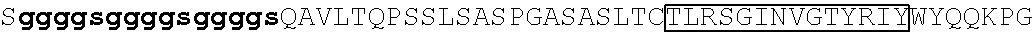

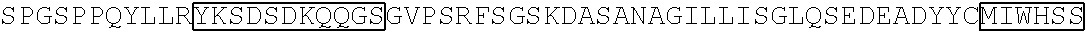

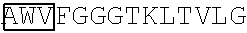

64
(SEQ ID NO: 8)

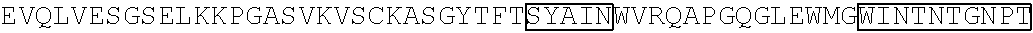

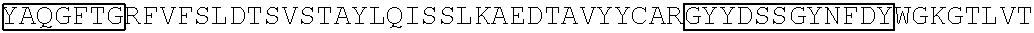

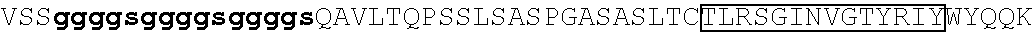

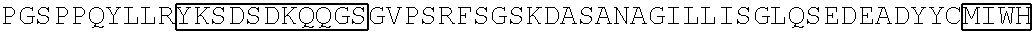

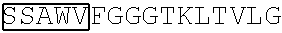

65 (scFv-5S)
(SEQ ID NO: 9)

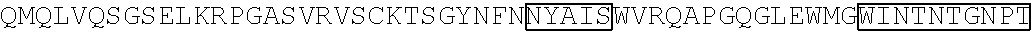

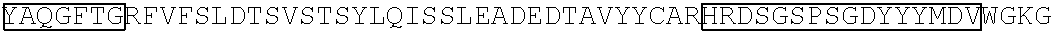

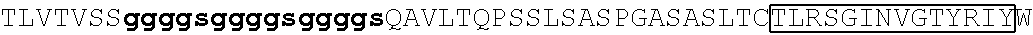

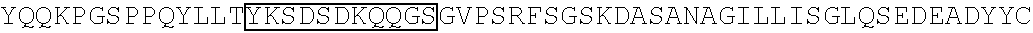

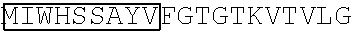

69
(SEQ ID NO: 10)

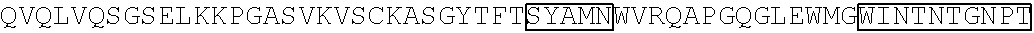

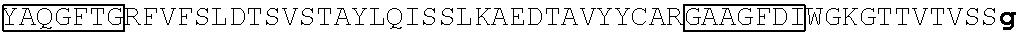

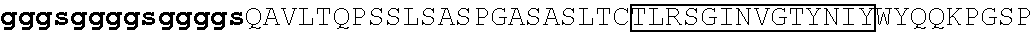

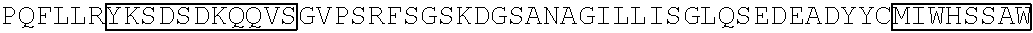

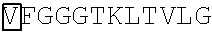

72
(SEQ ID NO: 11)

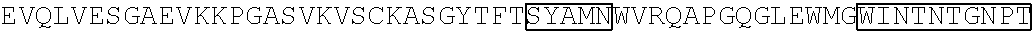

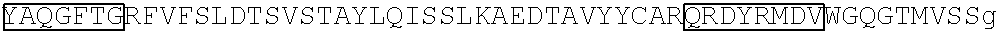

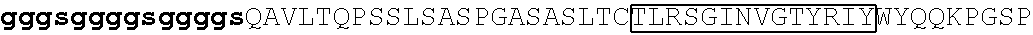

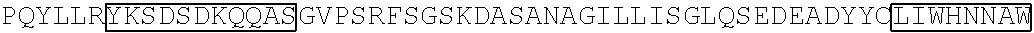

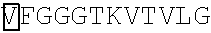

83
(SEQ ID NO: 12)

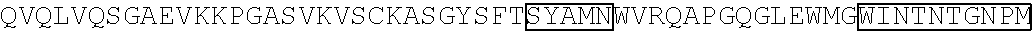

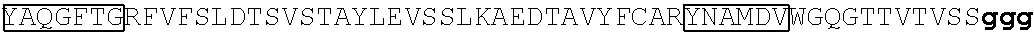

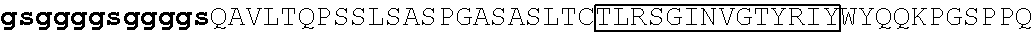

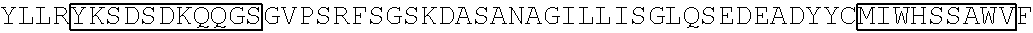

GGGTKVTVLG

Tables 2A-2B. CDR Sequence Alignments and Comparisons

TABLE 2A
V_{H} sequences

| Clone | | | | | | |
|---|---|---|---|---|---|---|
| No. | CDR1 | # | CDR2 | # | CDR3 | # |
| 18 | NYAMN | | WINTNTGNPTYAQGFTG | | GSLGG | |
| (1S) | | | | | TFDY | |
| 36 | SYGIS | | WINTNTGNPTYAQGFTG | | HSDDG | |
| (2S) | | | | | AFDI | |
| 44 | DYAIN | | WINTNTGNPTYAQGFTG | | HFSGYS | |
| (3S) | | | | | LDAVDI | |
| 51 | SYAMN | | WINTNTGNPTYAQGFTG | | FDRPRG | |
| (4S) | | | | | AFDI | |
| 65 | NYAIS | | WINTNTGNPTYAQGFTG | | HRDSGSPS | |
| (5S) | | | | | GDYYYMDV | |
| 64 | SYAIN | | WINTNTGNPTYAQGFTG | | GYYDSSGY | |
| | | | | | NFDY | |
| 69 | SYAMN | | WINTNTGNPTYAQGFTG | | GAAGVFDI | |
| 72 | SYAMN | | WINTNTGNPTYAQGFTG | | QRDYRMDV | |
| 83 | SYAMN | | WINTNTGNPMYAQGFTG | | YNAMDV | |

TABLE 2B
V_{L} sequences

| Clone | | | | | | |
|---|---|---|---|---|---|---|
| No. | CDR1 | # | CDR2 | # | CDR3 | # |
| 18 | TLRSGINVGTYRIY | | YKSDSDKHQDS | | MIWHSRAWV | |
| (1S) | | | | | | |
| 36 | TLRSGINVGTYRIY | | YKSDSDKQQGS | | AIWHSSTWV | |
| (2S) | | | | | | |
| 44 | TLRSDINVGAYRIY | | FKSDSDNYRGS | | AIWHSSTWV | |
| (3S) | | | | | | |
| 51 | TLRSGINVGTYRIY | | YKSDSDKQQGS | | MIWHSSAWV | |
| (4S) | | | | | | |
| 65 | TLRSGINVGTYRIY | | YKSDSDKQQGS | | MIWHSSAYV | |
| (5S) | | | | | | |
| 64 | TLRSGINVGTYRIY | | YKSDSDKQQGS | | MIWHSSAWV | |
| 69 | TLRSGINVGTYNIY | | YKSDSDKQQVS | | MIWHSSAWV | |
| 72 | TLRSGINVGTYRIY | | YKSDSDKQQAS | | LIWHNNAWV | |
| 83 | TLRSGINVGTYRIY | | YKSDSDKQQGS | | MIWHSSAWV | |

### Example 5. Evaluation of Expressed Candidate Anti-Click Product scFv Molecules

The nine selected scFv candidates from the Hu-ScL-6 library screens were expressed as isolated hexahistidine (6H) fusion proteins in E. coli by standard molecular biological procedures. Of these, two (72 and 83) failed to yield sufficient protein after repeat attempts, and were not pursued further. The scFvs 64 and 69, while yielding good protein preparations in terms of amounts and purities, were of relatively low binding activities. The remaining five candidates (1S-5S, as above) were characterized in more detail.

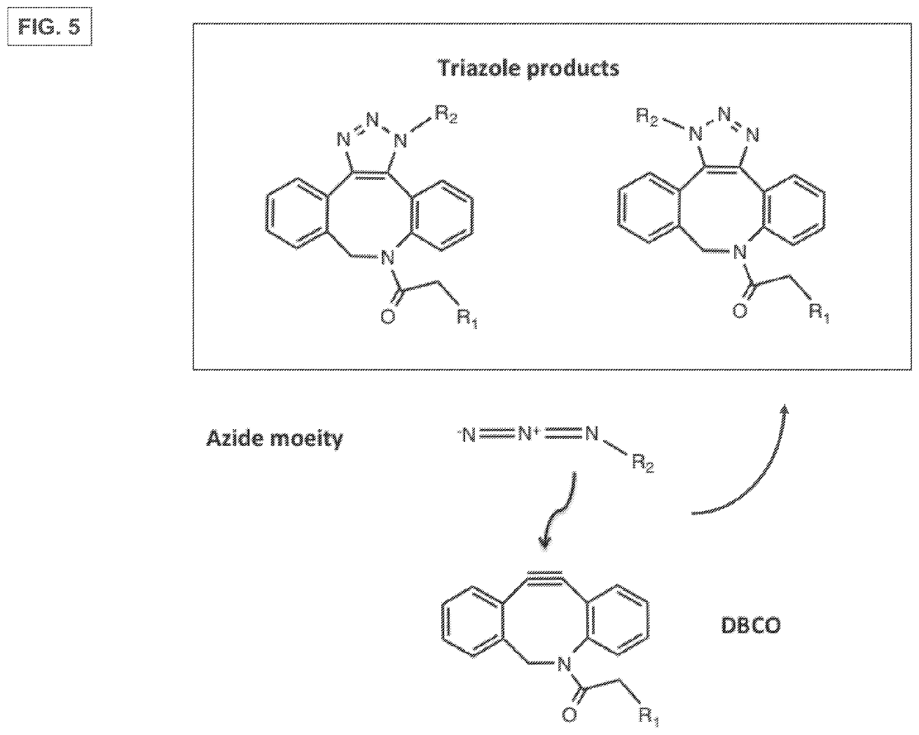

While the click peptides Az1db1 and Az2db1 were not used for selection of a click product-binding scFv from the second library, they were useful for screening of selected scFvs against Az3db1 for gauging the specificities of the their binding properties. It was initially found with phage-based ELISAs that all the anti-Az3db1 scFvs also cross-recognized the other two available click peptides. This suggested that the scFvs might be binding solely to the central core of the click peptides, which is comprised of the product of the reaction between DBCO and azide groups (FIG. 5). Accordingly, when the expressed scFv proteins 1S-5S were available, they were tested in ELISA assays against both the original target Az3db1 and also immobilized forms of DBCO and azide alone (flanked only by polyethylene glycol [PEG] groups), and the click products of these biotinylated reagents. It was found (FIG. 6; scFv proteins 1S and 2S examples), that while strong responses against Az3db1 were observed, no reactivity at all was seen towards either isolated azide or DBCO, nor their click product. The combined results suggested that while the nature of the peptide flanking sequences were not crucial, structures proximal to the central click core were important contributors to scFv recognition and binding affinity.

We ranked the five scFv proteins 1S-5S in terms of their desired specificities and binding affinities. For this purpose, a series of ELISA-based competition assays were established, where scFvs were preincubated in solution with designated competitors before testing against immobilized target Az3db1 peptide. These included Az3db1 itself, and its constituent peptides Az3 and db1, in large (×100-fold) molar excess over the scFv proteins.

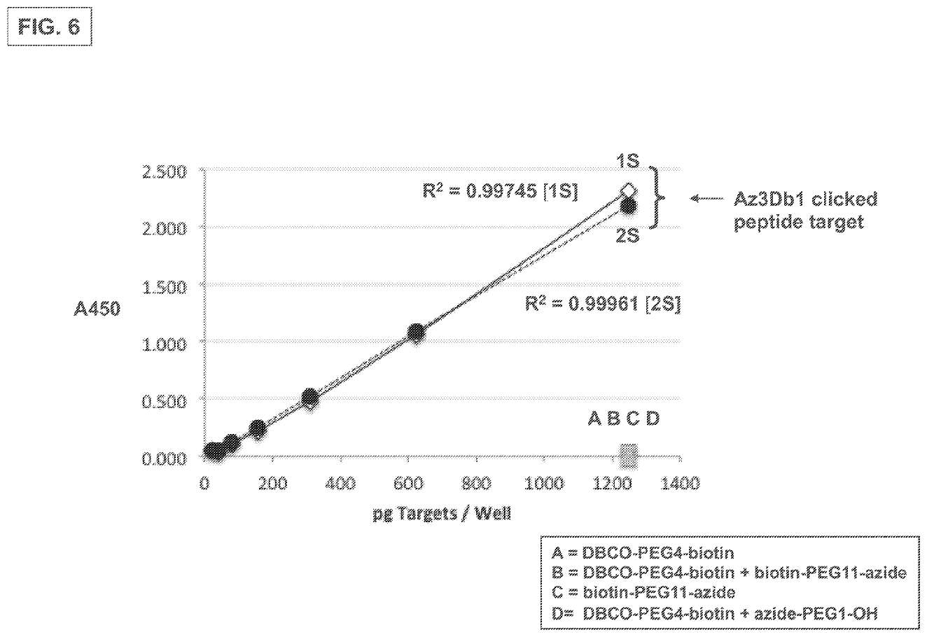

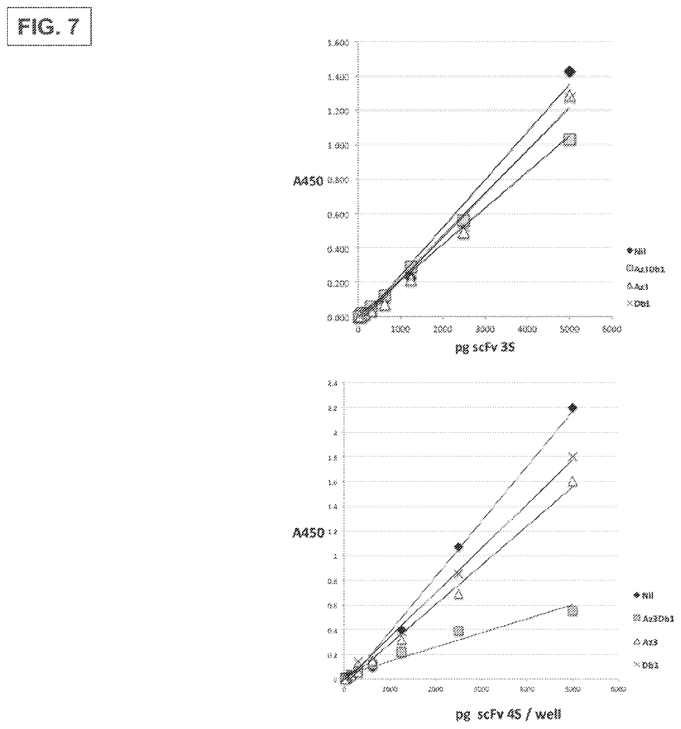

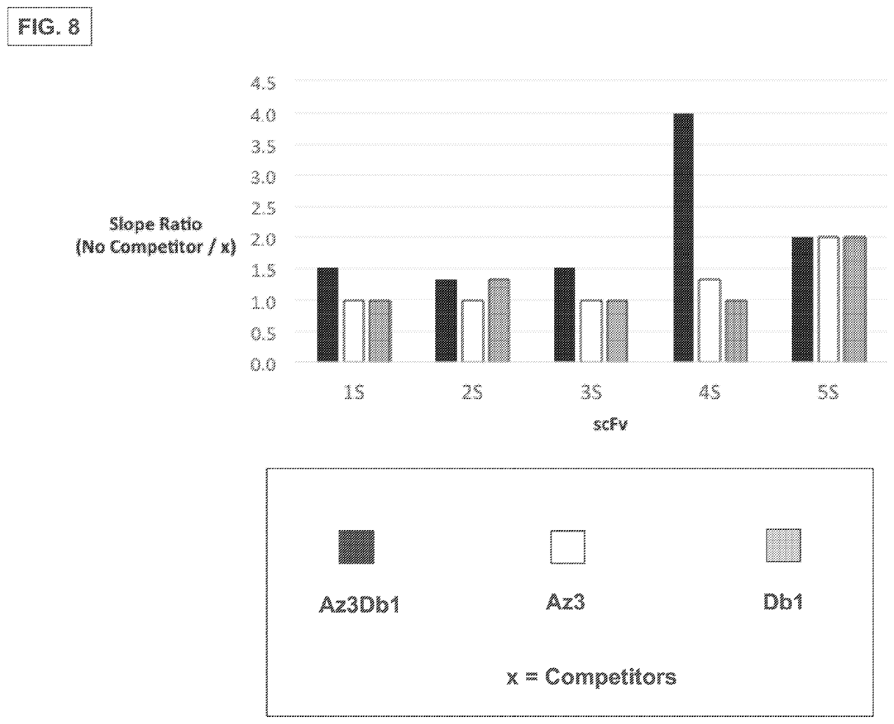

After incubation with a solution-phase candidate target, a molecule towards which the scFv has significant binding affinity will compete with binding to the solid-phase target in the ELISA plate, and thus reduce the slope of a signal curve (effectively reducing the concentration of available scFv for plate-target binding). The higher the affinity of an scFv or antibody for the solution-phase target, the greater the reduction in the observed response slope. Examples of this comparative slope assessment process are shown in FIG. 7 for two scFvs (3s and 4S), showing a marked difference for scFv 4S between the slopes with Az3db1 competitor vs. Az3 or db1 peptides alone. In contrast, this differential was much weaker for scFv 3S (FIG. 7). Ratios of slopes with and without competitors for the five expressed scFv proteins are shown in FIG. 8. Here a ratio of 1.0 for a given candidate competitor molecule indicated that no competition was observed—that is, the tested competitor in solution at high high molar excess had no appreciable effect on binding of the scFv to the solid-phase target. In turn, a high ratio is indicative of a competition effect, suggesting that the scFv had some binding affinity towards the competitor. As shown in FIG. 8, there was a high competition ratio for scFv 4S with Az3db1, very slight with Az3 alone, and none with db1 alone. The candidates scFvs 1S and 3S showed no competition with the single half-peptides, but very modest with the Az3db1 target. The scFv 2S showed no distinction in competition between the db1 half-peptide and Az3db1, while 5S gave higher ratios, but to equal degrees towards all of the competitors.

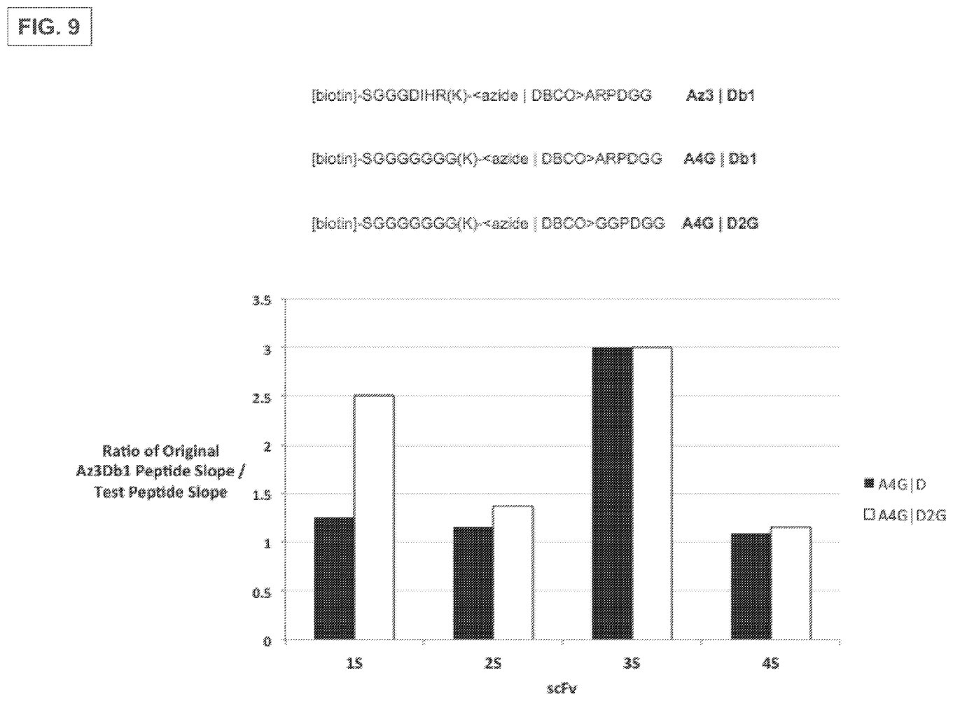

To extend the characterization of the scFv binding specificities, a series of click peptide analogs of Az3db1 were tested for scFv binding. For these, the flanking amino acid residues were successively replaced with glycines, on either side of the click-product chemical join. Results for two of these peptides with maximal glycine substitutions are shown in comparison with the original Az3db1 (FIG. 9). The slopes in ELISA assays against Az3db1 and the substituent peptides A4GDb1 and A4GD2G were measured at the same time for scFvs 1S-4S (5S was excluded from further assessment due to its unfavorable specificity properties (FIG. 8). For such assessment, a slope of 1.0 indicates equal efficacy towards both the original and the test altered click peptide; a slope of greater than 1.0 implies that the response to the test substituted peptide is inferior to the original selection peptide. (A slope of less than 1.0 would be equivalent to a ‘heteroclitic’ response, where the challenge target is superior to the original; not observed in this set). Of these four scFvs, it can be seen that 4S responses were close to 1.0; markedly distinct results were obtained for the remaining three. The scFv 1S was inferior in recognition towards the most completely substituted peptide A4GD2G, while 3S was inferior to both the latter and also A4GDb1. The scFv 2S gave better recognition of the substituted peptides over the latter two, but still not as good as 4S.

Based on these findings, the scFv 4S (scFv-51) was identified as possessing superior binding and specificity properties over the other evaluated candidates. The ability of 4S to bind glycine-substituted peptides (FIG. 9) but not isolated click products implied that it recognized both the click product (FIG. 5) and proximal flanking structures, without being constrained by the identity of flanking peptide residues.

### Example 6. Conversion of scFv-51 into Human IgG1 (IgG1-51)

By standard molecular biological methods, scFv-51 (4S) was converted into a full human antibody of γ1 heavy chain constant region isotype, and κ light chains, using the following sequences:
Human Gamma1 C-region Database Uniprot code P01857.1
Human Kappa C-region Database Uniproti code P01834.2

The relevant V_{H} and V_{L} sequences for scFv-51 were separately fused in-frame with downstream coding sequences for Cγ1 and Cκ, respectively. Sequences were separately cloned into the pcDNA3 mammalian expression vector and co-expressed in Hek293 cells.

### Example 7. Characterization of IgG1-51 Recognition Properties

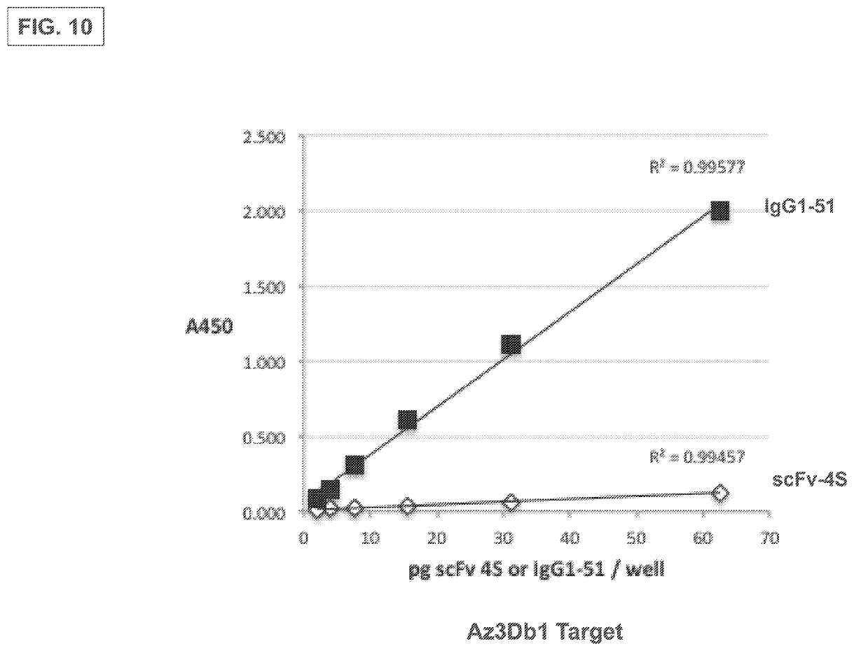

IgG1-51 was initially tested for its recognition of Az3db1 in comparison with the parental scFv-51 (4S) in a standard ELISA. It was found (FIG. 10) that the full antibody produced a robust response, markedly stronger than the original scFv. This recognition was highly reproducible. In common with scFv 4S, IgG1-51 also recognized the glycine-substituted peptide A4GD2G with equal facility.

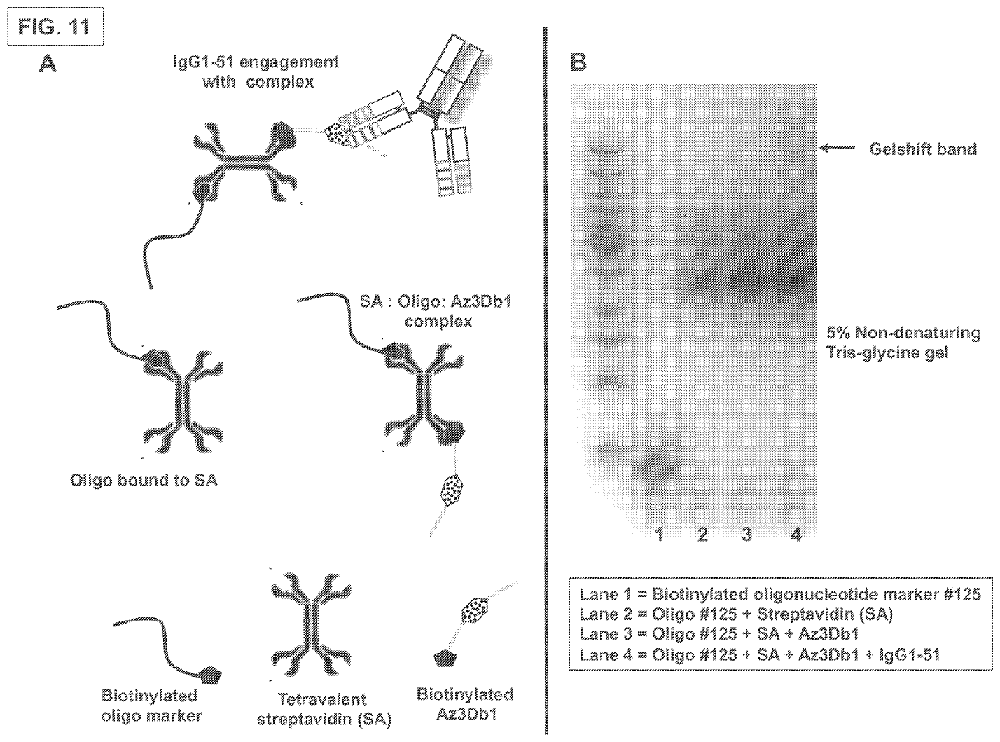

An independent means for demonstrating IgG1-51 target click peptide binding was devised in the form of a novel gel-shift assay exploiting the tetravalency of streptavidin (SA). For this, a biotinylated oligonucleotide was used as a stainable marker, in conjunction with the available biotinylated Az3db1 (FIG. 3). When either oligo alone or both oligo and click peptide are thus bound to soluble SA, a pronounced mobility retardation is observed on non-denaturing Tris-glycine gels (of low % acrylamide) relative to oligo alone (FIG. 11). The presence of >1 retardation band is consistent with the formation of complexes with different amounts of oligo vs. click peptide bound to SA. Addition of IgG1-51 resulted in a supershifted band, indicative of antibody interaction with the complex (FIG. 11).

### Example 8. Measuring IgG1-51 Binding Affinity Towards Az3db1 Target

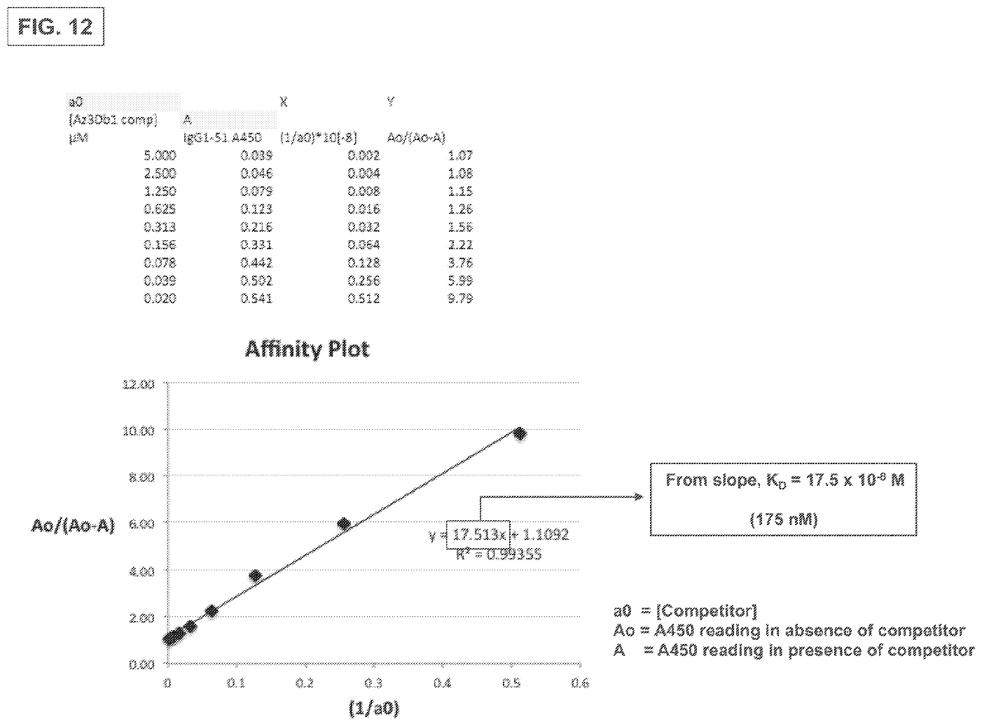

The binding affinity of IgG1-51 antibody towards Az3db1 was measured in an ELISA-based format by the method of Friguet et al. (1985), to yield the dissociation constant (K_{D}) from a specific data plot. An example of one such determination is presented in FIG. 12. Because the total concentration of free competitor in these studies is large compared to the concentration of antibody (antibody concentration ˜0.1 nm), the simplification of the Scatchard-Klotz equation as derived by Friguet et al. was used, as:

Ao/(Ao−A)=1+K_{D}(1/ao)
where Ao=absorbance (A₄₅₀) in the absence of competitor; A=absorbance in the presence of a specific competitor concentration; ao=concentration of competitor.

This equation conforms to the general linear graphic y=mx+c, where K_{D} thus corresponds to the slope of a derived plot for Ao/(Ao−A) vs. (1/ao).

From four independent determinations, the K_{D} value was averaged and determined to be 160+19 nM, thus placing the antibody recognition binding affinity in the nanomolar range.

### Example 9: ELISA Assay for scFv or IgG1-51 Activity Against Az3db1

All ELISA assays used biotinylated target peptides for immobilized on streptavidin (SA) plates. For testing responses against Az3db1 (FIG. 3), a stock of the click product between peptides Az3 and db1 was made by incubating both together at 4.5 mM concentration in PBS pH 7.4 for 16 hr, conditions resulting in ≥70% conversion to the click product (FIG. 4). The resulting Az3db1 was positioned in an SA plate by incubating appropriate wells (after initial plate washing) with 100 μl of 1 pmol/μl of peptide. Dilutions of scFvs or IgG1-51 were made in PBS with 10 mg/ml bovine serum albumin (BSA) to levels typically beginning at 50 pg/μl, then in extended 2-fold dilution series from that point. Antibody or scFv dilutions were then incubated with Az3db1 target for 1 hr/room temperature, followed by treatment with appropriate secondary antibody-HRP conjugates (anti-human kappa for IgG1-51, anti-6H hexahistidine for all scFvs). After extensive washing, signals based on HRP activity from standard chromogenic substrates were read in a plate reader and collated. FIG. 10 shows an example of such an assay read-out.

### Example 10: Binding of scFv 4S to Cells Metabolically Labeled with Surface Azide and Treated with Peptide db1

Biotin-(PEG)₁₁-azide was reacted with db1 under conditions of high reactant concentration in a similar manner as for other non-templated in vitro click reactions (Example 9). The product was immobilized in streptavidin (SA) plates, probed with scFv 4S, and treated with secondary anti-6H-HRP antibody in a comparable manner as in Example 9.

The scFv 4S did not significantly bind to the constituent peptides of Az3db1 (Az3 and db1) in isolation, although good recognition of glycine-substituted derivatives of Az3db1 is obtained (FIG. 9). Accordingly, it was useful to test if 4S could recognize a ‘half-peptide’ (one of the constituent click precursors of Az3db1) that had reacted with its complementary click group lacking a peptide flank. Thus, for the peptide db1 (DBCO-ARPDGG), the challenge target becomes the click product:

(chemical flank)-<azide-DBCO>-ARPDGG

This is equivalent to an azide-DBCO product core flanked on the former DBCO side by the same short peptide as for db1, with the former azide side flanked by the non-peptide chemical structure chosen to be immediately adjacent. With commercially available materials, a number of options exist, including alkyl groups of varying lengths. Polyethylene glycol (PEG) linkers of a wide range of repeat units are commonly used for their beneficial solubility-enhancing effects. A biotin-(PEG)₁₁-azide product (Broadpharm) was thus used for reaction with db1, where the biotin enables the immobilization of the product on streptavidin plates for subsequent standard ELISA testing.

It was found (FIG. 13), that scFv 4S showed significant reactivity towards the biotin-(PEG)₁₁-<azide-DBCO>db1 product, albeit much more weakly than towards Az3db1 itself. Other tested scFvs did not show this recognition of the half-peptide click product.

As an extension of this work, cells metabolically labeled with surface azides and treated with db1 peptide were tested for recognition by 4S scFv. Such azide modifications occur as terminal sugar residues of surface glycans, via sialic acid metabolic pathways utilizing introduced azidomannosamine. In such circumstances, the immediate ‘chemical flank’ for the db1 click product becomes:

(glycan chain)--(N-acetylmannosamine)-<azide-DBCO>-ARPDGG

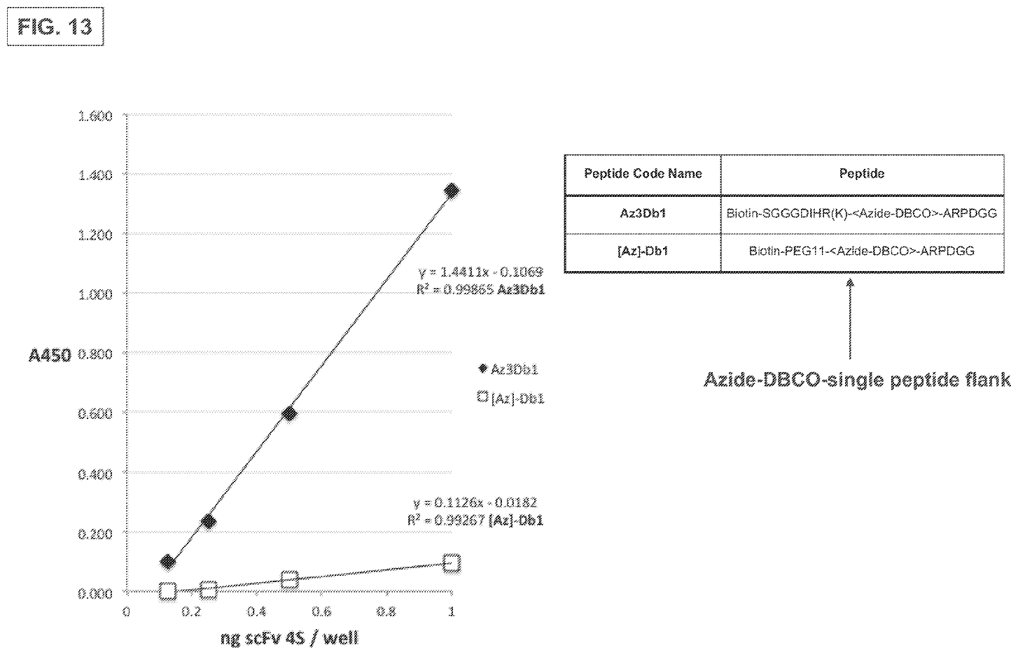

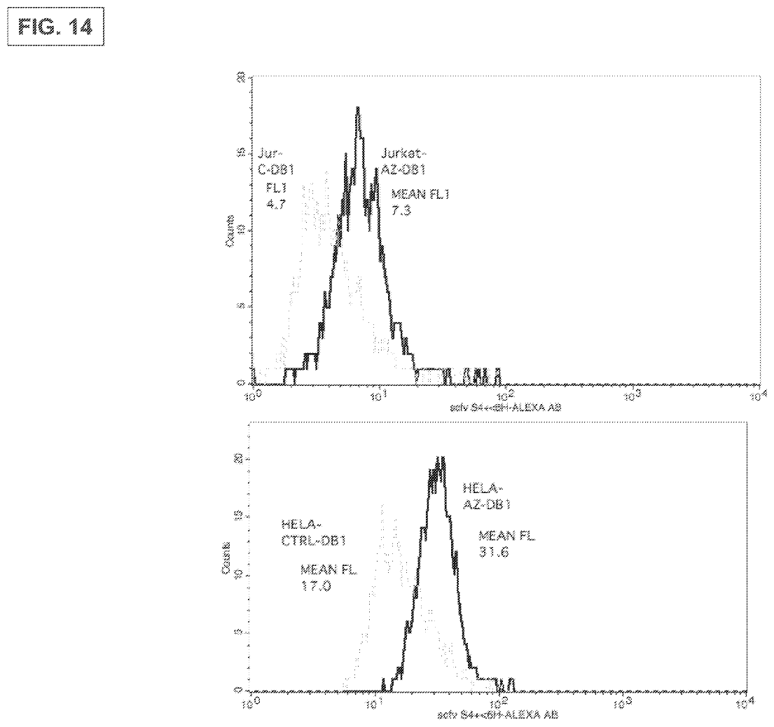

Following standard metabolic labeling of Jurkat and HeLa cells with AzNAM (acetylated azido-N-acetylmannosamine) or DMSO solvent as controls, cells were treated with db1 peptide, washed, and then incubated with scFv 4S. The secondary antibody was an anti-6H-Alexa-Fluor488 conjugate (R&D Systems), and the cells were tested by flow cytometry with settings as used for fluorescein. Compared with db1 peptide-treated controls that had not been pre-treated with AzNAM, both Jurkat and HeLa cells showed significant binding of 4S after db1 treatment (FIG. 14). The differential between the responses of control db1 vs. AzNAM/db1 was greater for HeLa cells.

### Example 11: Binding of IgG1-51 Antibody to Cells Metabolically Labeled with Surface Azide and Treated with Peptide db1

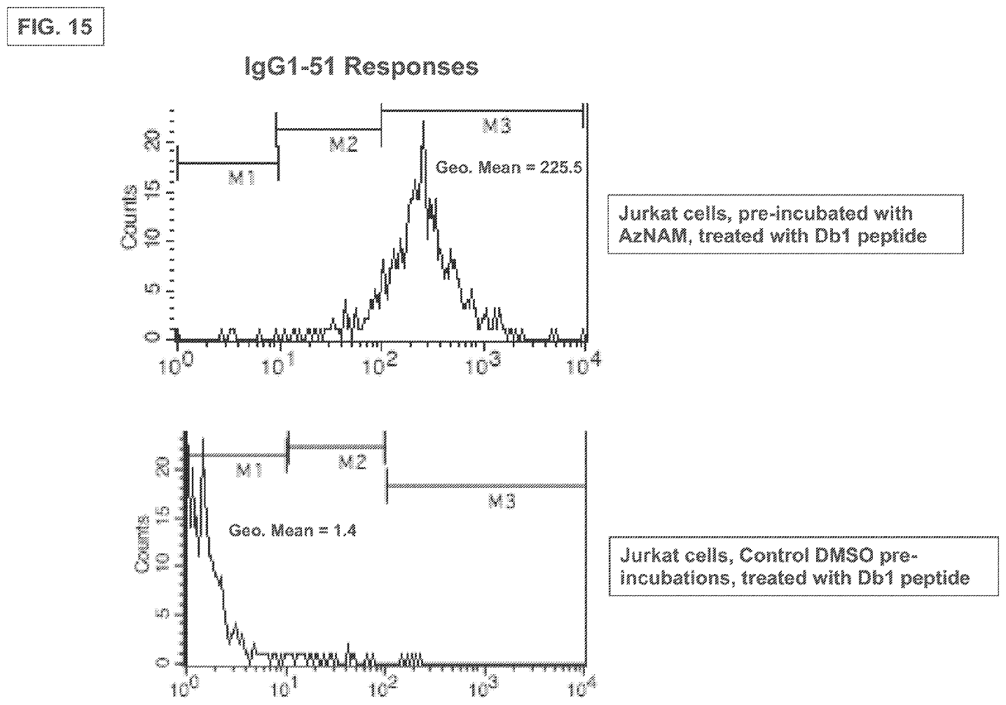

An analogous experiment to Example 2, above, was performed with the full antibody IgG1-51 derived from scFv 4S. Jurkat cells were treated with AzNAM or DMSO solvent control, and then after 22 hr treated in turn with peptide db1. After washing, cells were incubated with IgG1-51, followed by anti-human kappa chain—FITC as the secondary. Flow analysis revealed very significant staining of the db1-Jurkats (FIG. 15), markedly more than that seen with the same treatment and staining with scFv 4S (FIG. 14).

### Example 12: Templating of Az3 and db1 Peptide Derivative-Oligonucleotide Conjugate Haplomers on Solid-Phase or Cell-Surface Templates, and Recognition by IgG1-51

Peptides corresponding to Az3 and db1 (FIG. 3), or derivatives thereof (FIG. 9) are used to form conjugates with specifically reactive oligonucleotides. The chemistry in this application involves methyltetrazide (MTZ) and trans-cyclooctene (TCO), which are orthogonal click groups to azide and DBCO.

Thus, the following MTZ peptides are synthesized:

<MTZ>-SGGGERTH(K)-<azide>(MTZ-Az3)

<DBCO>-ARPDGG-<MTZ>(db1-MTZ)

In some embodiments, Az3 is replaced with the peptide A4G, and db1 is replaced with the peptide D2G (FIG. 9).

In addition, two TCO-labeled 2′-O-methyl oligonucleotides are prepared:

TCO-A 1:
(SEQ ID NO: 17)
5'-< TCO>UUUCUUCAGGACACAG.
B-TCO 2:
(SEQ ID NO: 18)
5'-GUCCAGAUGUCUUUGC <TCO>.

Oligo TCO-A is reacted with peptide MTZ-Az3, and oligo B-TCO is separately reacted with peptide db1, where both participating reactants are at millimolar concentrations (2-fold peptide molar excess) in phosphate buffer pH 7.0, for 12-16 hr. The degree of conversion of oligonucleotides is monitored by loading samples on a denaturing 8 M urea acrylamide gel, where TCO-MTZ click conjugates are readily tracked by their retarded mobilities relative to corresponding unreacted oligo alone. These peptide-oligonucleotide conjugates are now termed haplomers in accordance with TAPER terminology (see, e.g., WO2014197547), and are tested for templating and assembly of the DBCO-azide click product in an a ELISA format. Haplomer 1=oligo TCO-A:: peptide MTZ-Az3 conjugate; Hapomer 2=oligo B-TCO::peptide db1-MTZ conjugate.

The template (Template1) is a 40-mer 2′-O-methyl oligonucleotide with the sequence as shown:

Template1:
(SEQ ID NO: 19)
UUUUUCCUGUGUCCUGAAGAAAGCAAA
GACAUCUGGACAU

For ELISA screening of haplomers, Template1 is equipped with a 5′-biotin to enable its solid-phase placement in streptavidin (SA) plates.

SA plate wells are treated with 100 μl of 0.2 pmol/μl solution of biotinylated Template1, washed, and the tested with Haplomer 1 and 2 alone, or Haplomer 1+2 in combination. After an incubation of 1 hr at room temperature to allow both hybridization and proximity-induced click reactions, plates are washed and tested with IgG1-51, followed by anti-human kappa chain-HRP conjugate and standard color development with HRP substrates. Positive signal with Haplomer 1+2 but with neither alone is indicative of successful templating.

Following this, the templating reaction is performed on cell surfaces with tethered templates. To enable this, Template1 is modified with a chemically synthesized 5′-tag corresponding to the ligand for a specific cellular surface receptor. In this Example, Template1 is modified with a 5′-folate molecular tag, to enable its interaction and binding with surface folate receptors. Human HeLa cells and mouse ID8 cells are good expressors of the alpha-folate receptor. Target cells are grown for two weeks in low-folate RPMI medium to reduce receptor folate saturation, and then incubated with 100 μM folate-Template1 in serum-free low-folate RPMI medium. After washing cells, treatment with similar haplomer combinations as for the above ELISA test is performed (along with additional controls in the form of cells without added template), followed by washing and testing with IgG1-51. Levels of antibody binding are then assessed with flow analysis in the same manner as for Example 3, except that the secondary antibody is a goat human kappa-FITC conjugate. An analogous pattern of results as seen with the preliminary ELISA (positive flow signal for Haplomer 1+2 but negative for either Haplomer 1 or Haplomer 2 alone) indicates successful surface templating.

The deployment of the templating system and the click-product recognition IgG1-51 antibody for in vivo applications is demonstrated, where the antibody is tagged with either a highly cytotoxic chemical (for targeted cell killing), or a fluorescent/luminescent marker for identification and flagging of cells with surface receptors of interest.

In various embodiments, the cytotoxic tag may be, but is not limited to, emtansine or calicheamicin. The fluorescent tag may be a small fluorescent protein comprised of, but not limited to, superfolder GFP, mCherry, or dsRed. The luminescent tag may be composed of, but not limited to, Gaussia luciferase, Renilla luciferase, or Nanoluc® luciferase.

What is claimed is:

1. A click product binding molecule that specifically binds to a click product of structure (1):
[A]-<azide-dibenzocyclooctyne(DBCO)>-[B] (1),
where [A] and [B] comprise distinct sequences, e.g., distinct tetrapeptide, pentapeptide, hexapeptide, or heptapeptide sequences, wherein the antibody comprises a heavy chain variable region (VH) from an antibody described herein, e.g., Ab 51 as described herein, e.g., comprising a VH complementarity determining region (CDR)1 comprising an amino acid sequence set forth in Table 1A, a VH CDR2 comprising an amino acid sequence set forth in Table 1A, and a VH CDR3 comprising an amino acid sequence set forth in Table 1A; and a light chain variable region (VL) comprising a VL CDR1 from Ab 51 as described herein, e.g., comprising an amino acid sequence set forth in Table 1B a VL CDR2 comprising an amino acid sequence set forth in Table 1B, and a VL CDR3 comprising an amino acid sequence set forth in Table 1B (preferably wherein the CDRs are all from the same CDR definition).

2. The click product binding molecule of claim 1, wherein the VH comprises an amino acid sequence having at least 80% sequence identity to the amino acid sequence set forth in SEQ ID NO: 1.

3. The click product binding molecule of claim 1, wherein the VL comprises an amino acid sequence having at least 80% sequence identity to the amino acid sequence set forth in SEQ ID NO:2.

4. The click product binding molecule of claim 1, wherein the VH comprises an amino acid sequence having at least 80% sequence identity to the amino acid sequence set forth in SEQ ID NO: 1, and the VL comprises an amino acid sequence having at least 80% sequence identity to the amino acid sequence set forth in SEQ ID NO:2.

5. The click product binding molecule of claim 1, wherein the VH comprises the amino acid sequence set forth in SEQ ID NO:1.

6. The click product binding molecule of claim 1, wherein the VL comprises the amino acid sequence set forth in SEQ ID NO:2.

7. The click product binding molecule of claim 1, wherein the VH comprises the amino acid sequence set forth in SEQ ID NO: 1, and the VL comprises the amino acid sequence set forth in SEQ ID NO: 2.

8. The click product binding molecule of claim 1, wherein the VH consists of the amino acid sequence set forth in SEQ ID NO: 1.

9. The click product binding molecule of claim 1, wherein the VL consists of the amino acid sequence set forth in SEQ ID NO:2.

10. The click product binding molecule of claims 1 to 9, which is a single chain antibody molecule (scFv).

11. The click product binding molecule of claims 1-10, which comprises a heavy chain and a light chain and optionally an Fc region, preferably a human IgG1 Fc.

12. A single chain antibody molecule (scFv) that specifically binds to a click product of structure (1):
[A]-<azide-DBCO>-[B] (1),
where [A] and [B] comprise distinct sequences, comprising:
a VH consisting of the amino acid sequence set forth in SEQ ID NO:1 and a VL consisting of the amino acid sequence set forth in SEQ ID NO:2, or the amino acid sequence set forth in any one of SEQ ID NOs:4-12.

13. A polynucleotide comprising a nucleic acid sequence encoding the click product binding molecule of any one of claims 1-11, or the scFv of claim 12.

14. A polynucleotide comprising a nucleic acid sequence encoding a VH comprising the amino acid sequence set forth in SEQ ID NO: 1.

15. A polynucleotide comprising a nucleic acid sequence encoding VL comprising the amino acid sequence set forth in SEQ ID NO: 2.

16. The polynucleotide of claim 13 or 14, wherein the nucleic acid sequence is operably linked to a promoter.

17. A polynucleotide comprising a first nucleic acid sequence encoding a VH comprising the amino acid sequence set forth in SEQ ID NO: 1 and a second nucleic acid sequence encoding a VL comprising the amino acid sequence set forth in SEQ ID NO:2.

18. The polynucleotide of claim 16, wherein the first nucleic acid sequence is operably linked to a first promoter, and wherein the second nucleic acid sequence is operably linked to a second promoter.

19. A vector comprising the polynucleotide of any one of claims 13-18.

20. A vector comprising the polynucleotide of any one of claims 13-18.

21. A host cell comprising the polynucleotide of any one of claims 13-18.

22. A host cell comprising the vector of any one of claims 19-20.

23. A pharmaceutical composition comprising the click product binding molecule of any one of claims 1-11, or the scFv of claim 12, and a pharmaceutically acceptable carrier or diluent.

24. A method of making the click product binding molecule of any one of claims 1-11, or the ScFv of claim 12, comprising culturing the host cell of claim 21 and isolating the click product binding molecule or scFv.

25. The method of claim 24, further comprising formulating the antibody into a sterile pharmaceutical composition.

26. A method of detecting formation of a click product of structure (1):
[A]-<azide-DBCO>-[B] (1),
where [A] and [B] comprise distinct sequences, in a sample, the method comprising contacting the sample with the click product binding molecule of any one of claims 1-11, or the scFv of claim 12, and detecting binding of the click product binding molecule or scFv to the sample.

27. The method of claim 26, wherein the sample comprises:
haplomer A comprising at least a first sequence and an azide moiety; haplomer B comprising at least a second sequence and DBCO moiety; wherein the click product is produced is produced when haplomer A is in proximity with haplomer B.

28. The method of claim 27, wherein haplomer A and haplomer B bind to the same target.

29. The method of claim 27, wherein haplomer A and haplomer B bind to two different targets, wherein the two different targets can interact in the sample.
